(19) United States
(12) Patent Application Publication
(10) Pub. No.: US 2021/0145844 A1
(43) Pub. Date: May 20, 2021

(54) COMBINATION THERAPY FOR NICOTINE ADDICTION

(71) Applicant: Amygdala Neurosciences, Inc., San Francisco, CA (US)

(72) Inventor: Brent BLACKBURN, Los Altos, CA (US)

(73) Assignee: Amygdala Neurosciences, Inc., San Francisco, CA (US)

(21) Appl. No.: 17/157,058

(22) Filed: Jan. 25, 2021

Related U.S. Application Data
(63) Continuation of application No. PCT/US2019/043516, filed on Jul. 25, 2019.
(60) Provisional application No. 62/711,198, filed on Jul. 27, 2018.

(51) Int. Cl. A61K 31/55; A61K 45/06

(52) U.S. Cl. CPC A61K 31/55; A61K 45/06

(57) ABSTRACT

Disclosed is a combination therapy for reducing nicotine addiction or aiding in the cessation or lessening of tobacco use in a mammal, comprising administering to said mammal an amount of an ALDH-2 inhibitor, such as a compound of Formula (I), in combination with an amount of the nicotinic acetylcholine receptor agonist, varenicline. The disclosure further relates to methods and pharmaceutical compositions useful with the combination therapy.

### CROSS-REFERENCE

This application is continuation of PCT/US19/43516, filed Jul. 25, 2019, which claims priority to U.S. Provisional Patent Application No. 62/711,198, filed Jul. 27, 2018, each of which is hereby incorporated by reference herein for all purposes.

### FIELD

The present disclosure relates to a novel combination therapy for reducing nicotine addiction or aiding in the cessation or lessening of tobacco use in a mammal comprising administering to the mammal an amount of an aldehyde dehydrogenase-2 (ALDH-2) inhibitor in combination with the nicotinic acetylcholine receptor agonist compound, varenicline (CHANTIX®), whereby the combination acts to reduce nicotine addiction and aid in cessation or lessening of tobacco use by the mammal. The disclosure further relates to methods and pharmaceutical compositions useful with the combination therapy.

### BACKGROUND

Tobacco use remains a major health problem around the world. It is estimated that 1.1 billion people worldwide smoke tobacco. Although in North America smoking has been decreasing, smoking still is prevalent in the world's developing countries where it continues to rise. Human health problems resulting from tobacco use and the costs these health problems incur upon society are enormous. For example, tobacco smoking is the most common cause of cancer-related deaths and the leading cause of heart disease, emphysema, and bronchitis.

Selective inhibitors of aldehyde dehydrogenase-2 (ALDH-2), such as 2,6-dichloro-N-[4-(2-oxo-1,2-dihydro-pyridin-4-yl)-benzyl]-benzamide (compound (1)),

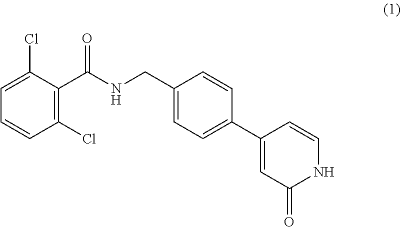 [SMILES: O=C(NCc1ccc(-c2cc[nH]c(=O)c2)cc1)c1c(Cl)cccc1Cl]

have been shown to suppress self-administration of nicotine in rats, as well as reduce intake of cocaine and alcohol. See e.g., U.S. Pat. Nos. 8,558,001, 8,575,353, 9,000,015, 9,610,299; Int'l Pat. Publ. WO2013/006400; and Rezvani et al., “Inhibition of Aldehyde Dehydrogenase-2 (ALDH-2) Suppresses Nicotine Self-Administration in Rats,” (2015) Journal of Drug and Alcohol Research, vol. 4: 1-6. Additionally, daidzein and several of its isoflavone structure related derivatives have been shown to selectively inhibit ALDH-2 and exhibit effectiveness treating alcohol dependency. See e.g., Keung et al., (1993) Proc. Natl. Acad. Sci. USA 90, 10008-10012; Keung et al., (1997) Proc. Natl. Acad. Sci. USA 94, 1675-1679; U.S. Pat. Nos. 5,624,910, 6,121,010, 7,951,813, 8,158,810, and 8,673,966; International Patent Publ. Nos. WO2008/014497, WO2008/124532, WO2009/061924, WO2009/094028, and WO2013/033377.

Studies have shown that ALDH-2 inhibition reduces pathophysiologic dopamine surge without changing basal dopamine levels. See e.g., Yao et al., “Inhibition of aldehyde dehydrogenase-2 suppresses cocaine seeking by generating THP, a cocaine use-dependent inhibitor of dopamine synthesis,” Nature Medicine (2010), Vol. 16, No. 9; Diamond and Yao, “From Ancient Chinese Medicine to a Novel Approach to Treat Cocaine Addiction,” CNS & Neurological Disorders—Drug Targets (2015) Vol. 14, No. 6. A recent review concludes that dopamine surge above normal levels is part of the reward circuit common to all drugs of addiction. See e.g., Volkow et al., “Neurobiologic Advances from the Brain Disease Model of Addiction,” N. Engl. J. Med. (2016) 374:363-371.

Varenicline, as the tartrate salt (7,8,9,10-tetrahydro-6,10-methano-6H-pyrazino[2,3-h][3]benzazepine, (2R,3R)-2,3-dihydroxybutanedioate(1:1)) is the active ingredient in CHANTIX™ which is marketed by Pfizer to aid adults in the cessation or lessening of smoking.

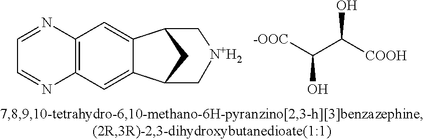

Varenicline binds with selectivity and high affinity to α4β2 neuronal nicotinic acetylcholine receptors and acts a partial agonist for these receptor subtypes. Electrophysiology studies have shown that varenicline binds to α4β2 neuronal nicotinic acetylcholine receptors and stimulates receptor-mediated activity, but at a level significantly lower than nicotine-induced stimulation. Additionally, varenicline blocks nicotine binding to the α4β2 receptors and thereby inhibits nicotine-induced stimulation of the central nervous mesolimbic dopamine system that is believed to be the neuronal mechanism underlying reinforcement and reward experienced upon smoking. For the treatment of nicotine addiction and/or as an aid in the cessation of tobacco use in humans, varenicline is administered orally as tablets containing 0.85 mg or 1.71 mg of varenicline tartrate salt, which is equivalent to 0.50 mg or 1.00 mg of varenicline. Methods for the preparation of varenicline compositions and their use in treatment are disclosed in e.g., U.S. Pat. Nos. 6,410,550, 6,890,927, and 7,265,119, each which is hereby incorporated by reference herein.

A number of side-effects have been identified as associated with the use of varenicline to aid in smoking cessation. The most common side effects include mild nausea (˜30% of patients), headaches, difficulty sleeping, and nightmares. Rarely reported side effects have included change in taste, vomiting, abdominal pain, flatulence, and constipation. The incidence of nausea was found to be higher at larger doses (30%) versus placebo (10%) relative to incidence at smaller doses (16%) versus placebo (11%). It is estimated that gastrointestinal side-effects lead to varenicline discontinuation in 2%-8% of patients. Additionally, in 2007 the FDA announced post-marketing reports that some patients using varenicline for smoking cessation experienced neuropsychiatric side-effects including thoughts of suicide, occasional suicidal behavior, erratic behavior, and drowsiness. In 2009, the FDA required varenicline to carry a warning that usage should be ceased if any of these symptoms are experienced. In June 2011, the FDA issued a safety announcement that varenicline may be associated with “a small, increased risk of certain cardiovascular adverse events in people who have cardiovascular disease.” Subsequent studies, systematic reviews, and meta-analyses, however, have failed to find evidence for increased risks of cardiovascular events, depression, or self-harm with varenicline versus nicotine replacement therapy. In 2016 the FDA removed the neuropsychiatric side-effect warning on varenicline, but patients are still advised to cease usage if they “notice any side effects on mood, behavior, or thinking.”

There remains a need for improved therapeutic compositions, formulations, and treatment methods with reduced side-effects and increased efficacy for aiding adults in the cessation or lessening of smoking.

### SUMMARY

As described above, varenicline has been shown to be effective as a therapeutic to aid adults in the cessation or lessening of smoking. Varenicline's efficacy results from its activity as a partial agonist of α4β2 receptors while also inhibiting nicotine-induced stimulation of the central nervous mesolimbic dopamine system. Selective ALDH-2 inhibitors, such as compounds of Formula (I), have been shown to be effective in suppressing nicotine self-administration in animal studies. It is believed that selective ALDH-2 inhibition reduces pathophysiologic dopamine surge without changing basal dopamine levels thereby effectively mitigating the reward circuit needed for addiction. The present disclosure provides improved therapeutic compositions and methods for aiding in the cessation or lessening of smoking in mammals, wherein the compositions and methods utilize varenicline in combination with an ALDH-2 inhibitor.

In some embodiments, the present disclosure provides methods for reducing nicotine addiction or aiding in the cessation or lessening of tobacco use in a mammal, comprising administering to said mammal a therapeutically effective amount of varenicline in combination with a therapeutically effective amount of an ALDH-2 inhibitor.

In some embodiments of the methods disclosed herein, the therapeutically effective amount of varenicline that is administered in combination with the ALDH-2 inhibitor comprises an amount of varenicline that is significantly lower than a therapeutically effective of varenicline when it is administered alone. Accordingly, in some embodiments of the methods the therapeutically effective amount of varenicline used in combination with the ALDH-2 inhibitor comprises an amount:

(a) less than 2.0 mg, less than 1.0 mg, less than 0.5 mg, less than 0.25 mg, less than 0.1 mg, or less than 0.05 mg;

(b) between about 0.05 mg and 2.0 mg, between about 0.05 mg and 1.0 mg, between about 0.05 mg and 0.5 mg, between about 0.05 mg and 0.4 mg, between about 0.05 mg and 0.25 mg, or between about 0.05 mg and 0.15 mg; or

(c) less than 2.0 mg/day, less than 1.0 mg/day, less than 0.5 mg/day, less than 0.25 mg/day, or less than 0.1 mg/day.

In some embodiments of the methods disclosed herein, the step of administering the varenicline in combination with the ALDH-2 inhibitor can comprise administering the varenicline and the ALDH-2 inhibitor separately. Optionally, administering separately can be selected from: (a) separately and not at the same time; or (b) separately and at the same time. In some embodiments, the ALDH-2 inhibitor is administered as a once-a-day dose.

In some embodiments of the methods disclosed herein, the varenicline and the ALDH-2 inhibitor are administered in a combination dosage form. In some embodiments the combination dosage form comprises a pharmaceutical composition of varenicline, the ALDH-2 inhibitor, and a pharmaceutically acceptable carrier. Optionally, the combination dosage form is an oral combination dosage form. In some embodiments, the combination dosage form comprises:

(a) less than 2.0 mg, less than 1.0 mg, less than 0.5 mg, less than 0.25 mg, less than 0.1 mg, or less than 0.05 mg of varenicline, and less than 5 mg, less than 2.5 mg, less than 1.0 mg, or less than 0.5 mg of the ALDH-2 inhibitor;

(b) less than 2.0 mg, less than 1.0 mg, between about 0.05 mg and 0.5 mg, between about 0.05 mg and 0.4 mg, between about 0.05 mg and 0.25 mg, or between about 0.05 mg and 0.15 mg of varenicline, and between 0.5 mg and 5 mg, between about 0.5 mg and 4.0 mg, between about 0.5 mg and 2.5 mg, or between about 0.5 mg and 1.5 mg of the ALDH-2 inhibitor; or

(c) less than 2.0 mg/day, less than 1.0 mg/day, 0.5 mg/day, less than 0.25 mg/day, or less than 0.1 mg/day of varenicline, and less than 5 mg/day, less than 2.5 mg/day, or less than 1.0 mg/day of the ALDH-2 inhibitor.

In some embodiments of the methods disclosed herein, the mammal is a human.

In some embodiments, the present disclosure provides a pharmaceutical composition comprising a therapeutically effective amount of varenicline, a therapeutically effective amount of an ALDH-2 inhibitor, and a pharmaceutically acceptable carrier.

In some embodiments, the pharmaceutical composition is formulated in a combination dosage form, optionally an oral combination dosage form. In some embodiments, the combination dosage form comprises:

(a) less than 2.0 mg, less than 1.0 mg, less than 0.5 mg, less than 0.25 mg, less than 0.1 mg, or less than 0.05 mg of varenicline, and less than 5 mg, less than 2.5 mg, less than 1.0 mg, or less than 0.5 mg of the ALDH-2 inhibitor;

(b) less than 2.0 mg, less than 1.0 mg, between about 0.05 mg and 0.5 mg, between about 0.05 mg and 0.4 mg, between about 0.05 mg and 0.25 mg, or between about 0.05 mg and 0.15 mg of varenicline, and between 0.5 mg and 5 mg, between about 0.5 mg and 4.0 mg, between about 0.5 mg and 2.5 mg, or between about 0.5 mg and 1.5 mg of the ALDH-2 inhibitor; or

(c) less than 2.0 mg/day, less than 1.0 mg/day, 0.5 mg/day, less than 0.25 mg/day, or less than 0.1 mg/day of varenicline, and less than 5 mg/day, less than 2.5 mg/day, or less than 1.0 mg/day of the ALDH-2 inhibitor.

In some embodiments, the pharmaceutical compositions disclosed herein are for use in therapy. In some embodiments, the disclosure provides for the use of the pharmaceutical compositions disclosed herein for the manufacture of a medicament, wherein the medicament is for reducing nicotine addiction or aiding in the cessation or lessening of tobacco use.

In the various embodiments of the methods and/or pharmaceutical compositions disclosed herein, the ALDH-2 inhibitor is a compound of Formula (I)

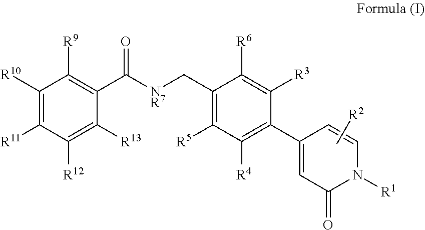

wherein:

R¹ is hydrogen, optionally substituted C₁₋₆ alkyl, —CH₂OH, —CH₂OP(O)(OR²⁰)(OR²¹);

R² is hydrogen, optionally substituted C₁₋₆ alkyl, cycloalkyl, or halo;

each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O—(C₁ to C₆-alkyl)-O—(C₁ to C₆-alkyl), cyano, halo, —SO₂NR²⁴R²⁵; or —NR²⁴R²⁵;

R⁷ is hydrogen or optionally substituted C₁₋₆ alkyl;

each of R²⁰ and R²⁴ is independently Na⁺, Li⁺, K⁺, hydrogen, C₁₋₆ alkyl; or R²⁰ and R²¹ can be combined to represent a single divalent cation Zn²⁺, Ca²⁺, or Mg²⁺; and

each of R²⁴ and R²⁵ is independently chosen from hydrogen or C₁₋₆ alkyl or when combined together with the nitrogen to which they are attached form a heterocycle; or

a pharmaceutically acceptable salt, ester, single stereoisomer, mixture of stereoisomers, or a tautomer thereof.

In some embodiments of the methods and/or pharmaceutical compositions disclosed herein, the ALDH-2 inhibitor is a compound the compound of formula (I) is selected from the group consisting of: 2,6-dichloro-4-(2-methoxyethoxy)-N-(4-(2-oxo-1,2-dihydropyridin-4-yl) benzyl)benzamide; 2,6-dichloro-N-[4-(2-oxo-1,2-dihydro-pyridin-4-yl)-benzyl]-benzamide; 2-chloro-3-fluoro-N-(4-(2-oxo-1,2-dihydropyridin-4-yl)benzyl)benzamide; 2-chloro-6-methyl-N-(4-(2-oxo-1,2-dihydropyridin-4-yl)benzyl)benzamide; 2,6-dimethyl-N-(4-(2-oxo-1,2-dihydropyridin-4-yl)benzyl)benzamide; 2,6-dichloro-N-[4-(6-methyl-2-oxo-1,2-dihydro-pyridin-4-yl)-benzyl]-benzamide; 2-chloro-3,6-difluoro-N-(4-(2-oxo-1,2-dihydropyridin-4-yl)benzyl)benzamide; 2,6-dichloro-N-(3-methyl-4-(2-oxo-1,2-dihydropyridin-4-yl)benzyl)benzamide); 2,6-dichloro-N-(4-(1-methyl-2-oxo-1,2-dihydropyridin-4-yl)benzyl)benzamide; 2,6-difluoro-N-(4-(2-oxo-1,2-dihydropyridin-4-yl)benzyl)benzamide; 2-chloro-6-fluoro-N-(4-(2-oxo-1,2-dihydropyridin-4-yl)benzyl)benzamide; 2,6-dichloro-N-(2-fluoro-4-(2-oxo-1,2-dihydropyridin-4-yl)benzyl)benzamide; 2,6-dichloro-N-(4-(5-fluoro-2-oxo-1,2-dihydropyridin-4-yl)benzyl)benzamide; phosphoric acid mono-(4-{4-[(2,6-dichloro-benzoylamino)-methyl]-phenyl}-2-oxo-2H-pyridin-1-ylmethyl) ester; 2,6-dimethyl-N-(4-(2-oxopiperidin-4-yl)benzyl)benzamide; or a pharmaceutically acceptable salt, single stereoisomer, mixture of stereoisomers, or a tautomer thereof.

In some embodiments of the methods and/or pharmaceutical compositions disclosed herein, the ALDH-2 inhibitor is a compound of formula (I), wherein the compound of formula (I) is compound (1):

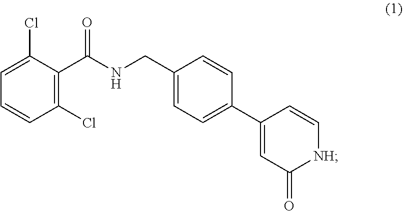 [SMILES: O=C(NCc1ccc(-c2cc[nH]c(=O)c2)cc1)c1c(Cl)cccc1Cl]

or a pharmaceutically acceptable salt, or a tautomer thereof.

In some embodiments of the methods and/or pharmaceutical compositions disclosed herein, the ALDH-2 inhibitor is a compound of formula (I), wherein the compound of formula (I) is compound (2):

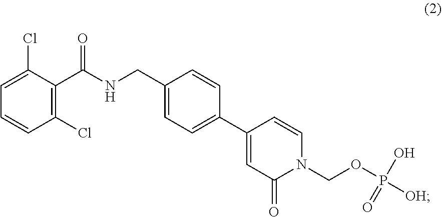 [SMILES: O=C(NCc1ccc(-c2ccn(COP(=O)(O)O)c(=O)c2)cc1)c1c(Cl)cccc1Cl]

or a pharmaceutically acceptable salt, ester, or a tautomer thereof.

In some embodiments of the methods and/or pharmaceutical compositions disclosed herein, the ALDH-2 inhibitor is a compound comprising an isoflavone structure. In some embodiments, the compound comprising an isoflavone structure is daidzein (compound (15)):

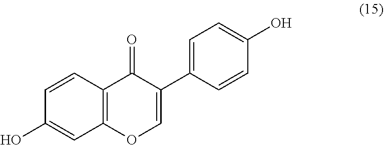 [SMILES: O=c1c(-c2ccc(O)cc2)coc2cc(O)ccc12]

or a pharmaceutically acceptable salt, ester, or a tautomer thereof. In some embodiments, the compound comprising an isoflavone structure is 3-{[3-(4-aminophenyl)-4-oxochromen-7-yloxy]methyl}benzoic acid (compound (16)):

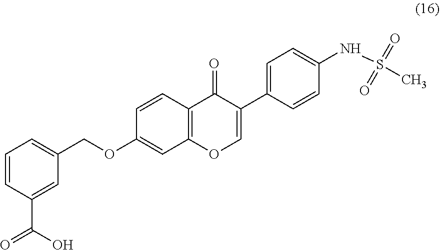 [SMILES: CS(=O)(=O)Nc1ccc(-c2coc3cc(OCc4cccc(C(=O)O)c4)ccc3c2=O)cc1]

or a pharmaceutically acceptable salt, ester, or a tautomer thereof.

Additionally, the present disclosure also provides a patient pack comprising at least one pharmaceutical composition as disclosed herein, and an information package or product insert containing directions on the method of using the pharmaceutical compositions.

Additional embodiments are described herein.

### DETAILED DESCRIPTION

It is to be understood that the detailed descriptions provided herein, including the drawings, are exemplary and explanatory only and are not restrictive of this disclosure. The description is not limited to the specific compounds, compositions, methods, techniques, protocols, cell lines, assays, and reagents disclosed herein, as these may vary, but is also intended to encompass known variants of these specific embodiments.

It is also to be understood that the terminology used herein is intended to describe particular embodiments and is in not intended to limit the scope as set forth in the appended claims. For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Further, it is understood that where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50” includes “2 to 25”, “5 to 20”, “25 to 50”, “1 to 10”, etc.

### ABBREVIATIONS, DEFINITIONS AND GENERAL PARAMETERS

As used in the present specification, the following words and phrases are generally intended to have the meanings as set forth below, except to the extent that the context in which they are used indicates otherwise.

The term “varenicline” as used herein includes 7,8,9,10-tetrahydro-6,10-methano-6H-pyrazino[2,3-h][3]benzazepine, as a free base, or as its tartrate salt, 7,8,9,10-tetrahydro-6,10-methano-6H-pyrazino[2,3-h][3]benzazepine, (2R,3R)-2,3-dihydroxybutanedioate(1:1), which is the active ingredient in CHANTIX™ a drug marketed by Pfizer to aid in smoking cessation. Additionally, “varenicline,” as used herein, is intended to include any pharmaceutically acceptable formulations of 7,8,9,10-tetrahydro-6,10-methano-6H-pyrazino[2,3-h][3]benzazepine including a single stereoisomer, a mixture of stereoisomers, one or more tautomers, or pharmaceutically acceptable salts other than tartrate.

The term “ALDH-2 inhibitor” as used herein includes any compound that selectively inhibits the enzyme aldehyde dehydrogenase 2. Exemplary ALDH-2 inhibitor compounds include the isoflavone compound, daidzein (see e.g., U.S. Pat. Nos. 5,624,910, and 6,121,010), and its structurally related isoflavone derivative compounds (see e.g., U.S. Pat. Nos. 7,951,813, 8,158,810, and 8,673,966; Int'l Pat. Publ. Nos. WO2008/014497, WO2008/124532, WO2009/061924, WO2009/094028, and WO2013/033377), and compounds of Formula (I), which are structurally unrelated to the isoflavones, such as 2,6-dichloro-N-[4-(2-oxo-1,2-dihydro-pyridin-4-yl)-benzyll-benzamide (see e.g., U.S. Pat. Nos. 8,558,001, 8,575,353, 9,000,015, 9,610,299; Int'l Pat. Publ. WO2013/006400).

The term “addiction” as used herein includes any substance use disorder including, but not limited to, substance misuse, substance dependence, substance addiction, and/or conditioned response behavior in a mammal resulting from a dopamine producing agent.

The term “dopamine producing agents” as used herein includes compounds capable of inducing a surge in dopamine levels in a mammal, including, but not limited to, opioids, amphetamines, alcohol, other drugs of addiction, foods (e.g., sugary foods), and nicotine.

The term “therapeutically effective amount” refers to an amount that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “unit dosage form” refers to physically discrete units suitable as unitary dosages for human subjects and other mammals, each unit containing a predetermined quantity of active ingredient that produces the desired therapeutic effect, in association with a suitable pharmaceutical excipient (e.g., a tablet, capsule, or ampoule).

The term “active ingredient” refers to a compound in a pharmaceutical composition that has a pharmacological effect when administered to an organism (e.g., a mammal) and is intended to encompass not only the compound but also the pharmaceutically acceptable salts, pharmaceutically acceptable esters, hydrates, polymorphs, and prodrugs of such compound.

The term “prodrug” refers to a compound that includes a chemical group which, in vivo, can be converted and/or split off from the remainder of the molecule to provide for the active drug, a pharmaceutically acceptable salt thereof, or a biologically active metabolite thereof.

The term “combination dosage form” refers to a unit dosage form (e.g., single pill, tablet, capsule, ampoule, suppository, or other unit dosage form) that contains a combination of two or more active ingredients (e.g., ALDH-2 inhibitor and varenicline).

The term “treatment” or “treating” means any administration of a compound of the disclosure to a mammal having a disease or disorder, or a mammal susceptible to a disease or disorder, for purposes including:
(i) preventing the disease, that is, causing the clinical symptoms of the disease not to develop;
(ii) inhibiting the disease, that is, arresting the development of clinical symptoms; and/or
(iii) relieving the disease, i.e. causing the regression of clinical symptoms.

The term “in combination with” as used in the context of administering the two or more active ingredients in a method of treatment (e.g., the varenicline and the ALDH-2 inhibitor compound) includes administering the active ingredients separately (e.g., sequentially) or together (e.g., simultaneously).

The term “alkyl” refers to a monoradical branched or unbranched saturated hydrocarbon chain having from 1 to 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
(i) an alkyl group as defined above, having 1, 2, 3, 4 or 5 substituents, (typically 1, 2, or 3 substituents) selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(ii) an alkyl group as defined above that is interrupted by 1-10 atoms (e.g. 1, 2, 3, 4, or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2; or
(iii) an alkyl group as defined above that has both 1, 2, 3, 4 or 5 substituents as defined above and is also interrupted by 1-10 atoms (e.g. 1, 2, 3, 4, or 5 atoms) as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents (typically 1, 2, or 3 substituents), as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, typically having from 1 to 20 carbon atoms (e.g. 1-10 carbon atoms, or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like.

The term “lower alkylene” refers to a diradical of a branched or unbranched saturated hydrocarbon chain, typically having 1, 2, 3, 4, 5, or 6 carbon atoms.

The term “substituted alkylene” refers to:
(i) an alkylene group as defined above having 1, 2, 3, 4, or 5 substituents (typically 1, 2, or 3 substituents) selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, SO₂ alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(ii) an alkylene group as defined above that is interrupted by 1-10 groups (e.g. 1, 2, 3, 4, or 5 groups) independently chosen from —O—, —S—, sulfonyl, —C(O)—, —C(O)O—, —C(O)N—, and —NR^{a}, where R^{a} is chosen from hydrogen, optionally substituted alkyl, cycloalkyl, cycloalkenyl, aryl, heteroaryl and heterocyclyl; or
(iii) an alkylene group as defined above that has both 1, 2, 3, 4 or 5 substituents as defined above and is also interrupted by 1-10 groups as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers(—CH₂CH(CO₂H)CH₂—), ethoxyethyl (—CH₂CH₂O—CH₂CH₂—), ethylmethylaminoethyl (—CH₂CH₂—N(CH₃)—CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group —O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkoxy” refers to the group R—O—, where R is optionally substituted alkyl or optionally substituted cycloalkyl, or R is a group —Y—Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are alkyl-O— and include, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

The term “lower alkoxy” refers to the group R—O— in which R is optionally substituted lower alkyl as defined above. This term is exemplified by groups such as methoxy, ethoxy, n-propoxy, iso-propoxy, n-butoxy, iso-butoxy, t-butoxy, n-hexyloxy, and the like.

The term “alkylthio” refers to the group R—S—, where R is as defined for alkoxy.

The term “alkenyl” refers to a monoradical of a branched or unbranched unsaturated hydrocarbon group typically having from 2 to 20 carbon atoms (more typically from 2 to 10 carbon atoms, e.g. 2 to 6 carbon atoms) and having from 1 to 6 carbon-carbon double bonds, e.g. 1, 2, or 3 carbon-carbon double bonds. Typical alkenyl groups include ethenyl (or vinyl, i.e. —CH═CH₂), 1-propylene (or allyl, —CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “alkynyl” refers to a monoradical of an unsaturated hydrocarbon, typically having from 2 to 20 carbon atoms (more typically from 2 to 10 carbon atoms, e.g. 2 to 6 carbon atoms) and having from 1 to 6 carbon-carbon triple bonds e.g. 1, 2, or 3 carbon-carbon triple bonds. Typical alkynyl groups include ethynyl (—C≡CH), propargyl (or propynyl, —C≡CCH₃), and the like. In the event alkynyl is attached to nitrogen, the triple bond cannot be alpha to the nitrogen.

The term “substituted alkynyl” refers to an alkynyl group as defined above having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, cycloalkyl, aryl, heteroaryl, heterocyclyl or where both R groups are joined to form a heterocyclic group (e.g., morpholino). Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “ester” or “carboxyester” refers to the group —C(O)OR, where R is alkyl, cycloalkyl, aryl, heteroaryl, or heterocyclyl, which may be optionally further substituted by alkyl, alkoxy, halogen, CF₃, amino, substituted amino, cyano, or —S(O)ₙR^{a}, in which R^{a} is alkyl, aryl, or heteroaryl and n is 0, 1 or 2.

The term “acylamino” refers to the group —NRC(O)R where each R is independently hydrogen, alkyl, aryl, heteroaryl, or heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2.

The term “acyloxy” refers to the groups —OC(O)-alkyl, —OC(O)-cycloalkyl, —OC(O)-aryl, —OC(O)-heteroaryl, and —OC(O)-heterocyclyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl, fluorenyl, and anthryl). Typical aryls include phenyl, fluorenyl, naphthyl, anthryl, and the like.

Unless otherwise constrained by the definition for the aryl substituent, such aryl groups can optionally be substituted with 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, aryl, heteroaryl and heterocyclyl provided that both R groups are not hydrogen, or a group —Y—Z, in which Y is optionally substituted alkylene and Z is alkenyl, cycloalkenyl, or alkynyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙ—R, where R is alkyl, aryl, or heteroaryl and n is 0, 1 or 2.

The term “carboxyalkyl” refers to the groups —C(O)O-alkyl, —C(O)O-cycloalkyl, where alkyl and cycloalkyl are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and bicyclo[2.2.1]heptane, or cyclic alkyl groups to which is fused an aryl group, for example indan, and the like.

The term “cycloalkenyl” refers to cyclic alkyl groups of from 3 to 20 carbon atoms having a single cyclic ring or multiple condensed rings and having at least one double bond and preferably from 1 to 2 double bonds.

The terms “substituted cycloalkyl” and “susbstituted cycloalkenyl” refer to cycloalkyl or cycloalkenyl groups having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. The term “substituted cycloalkyl” also includes cycloalkyl groups wherein one or more of the annular carbon atoms of the cycloalkyl group is a carbonyl group (i.e. an oxygen atom is oxo to the ring). Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “alkoxycarbonylamino” refers to a group —NHC(O)OR in which R is optionally substituted alkyl.

The term “alkyl amine” refers to R—NH₂ in which R is optionally substituted alkyl.

The term “dialkyl amine” refers to R—NHR in which each R is independently an optionally substituted alkyl.

The term “trialkyl amine” refers to NR₃ in which R each R is independently an optionally substituted alkyl.

The term “azido” refers to a group

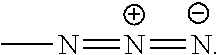

The term “hydroxyl” or “hydroxyl” refers to a group —OH.

The term “arylthio” refers to the group —S-aryl.

The term “heterocyclylthio” refers to the group —S-heterocyclyl.

The term “alkylthio” refers to the group —S-alkyl.

The term “aminosulfonyl” refers to the group —SO₂NRR, wherein each R is independently selected from the group consisting of hydrogen, alkyl, cycloalkyl, aryl, heteroaryl and heterocyclyl. Unless otherwise constrained by the definition, all substituents may optionally be further substituted by 1, 2, or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl.

The term “aminocarbonylamino” refers to the group —NR^{c}C(O)NRR, wherein R^{c} is hydrogen or alkyl and each R is independently selected from the group consisting of hydrogen, alkyl, cycloalkyl, aryl, heteroaryl and heterocyclyl. Unless otherwise constrained by the definition, all substituents may optionally be further substituted by 1, 2, or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl.

The term “heterocyclooxy” refers to the group —O-heterocyclyl.

The term “alkoxyamino” refers to the group —NHOR in which R is optionally substituted alkyl.

The term “hydroxyamino” refers to the group —NHOH.

The term “heteroaryl” refers to a group comprising single or multiple rings comprising 1 to 15 carbon atoms and 1 to 4 heteroatoms selected from oxygen, nitrogen, and sulfur within at least one ring. The term “heteroaryl” is generic to the terms “aromatic heteroaryl” and “partially saturated heteroaryl.” The term “aromatic heteroaryl” refers to a heteroaryl in which at least one ring is aromatic. Examples of aromatic heteroaryls include pyrrole, thiophene, pyridine, quinoline, pteridine. The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, and the like.

Unless otherwise constrained by the definition for the heteroaryl substituent, such heteroaryl groups can be optionally substituted with 1 to 5 substituents (typically 1, 2, or 3 substituents) selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl (an alkyl ester), arylthio, heteroaryl, heteroarylthio, heterocyclylthio, thiol, alkylthio, aryl, aryloxy, aralkyl, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2. Such heteroaryl groups can have a single ring (e.g., pyridyl or furyl) or multiple condensed rings (e.g., indolizinyl, benzothiazole, or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “heterocyclyl,” “heterocycle,” or “heterocyclic” refers to a monoradical saturated group having a single ring or multiple condensed rings, having from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2. Preferred heterocyclics include tetrahydrofuranyl, morpholino, piperidinyl, and the like.

The term “thiol” refers to the group —SH.

The term “substituted alkylthio” refers to the group —S-substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl. “Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl. “Substituted sulfone” refers to a group —S(O)₂R, in which R is substituted alkyl, substituted aryl, or substituted heteroaryl, as defined herein.

The term “keto” or “oxo” refers to a group —C(O)—.

The term “thiocarbonyl” refers to a group —C(S)—.

The term “carboxy” refers to a group —C(O)—OH.

The term “optional” or “optionally” mean that the subsequently described event or circumstance may or may not occur, and that the description includes instances where said event or circumstance occurs and instances in which it does not.

The term “substituted” includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to substituted aryl-(substituted aryl)-substituted aryl.

A compound of a given formula (e.g. the “compound of Formula (I)”) is intended to encompass the compounds of the disclosure, and the pharmaceutically acceptable salts, pharmaceutically acceptable esters, hydrates, polymorphs, and prodrugs of such compounds.

Additionally, the compounds of the disclosure may possess one or more asymmetric centers and can be produced as a racemic mixture or as individual enantiomers or diastereoisomers. The number of stereoisomers present in any given compound of a given Formula depends upon the number of asymmetric centers present (there are 2n stereoisomers possible where n is the number of asymmetric centers). The individual stereoisomers may be obtained by resolving a racemic or non-racemic mixture of an intermediate at some appropriate stage of the synthesis, or by resolution of the compound by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

The term “isomers” means different compounds that have the same molecular formula. Isomers include stereoisomers, enantiomers, and diastereomers.

The term “stereoisomers” means isomers that differ only in the way the atoms are arranged in space.

The term “enantiomers” means a pair of stereoisomers that are non-superimposable mirror images of each other. A 1:1 mixture of a pair of enantiomers is a “racemic” mixture. The term “(±)” is used to designate a racemic mixture where appropriate.

The term “diastereoisomers” means stereoisomers that have at least two asymmetric atoms, but which are not mirror-images of each other.

Absolute stereochemistry is specified herein according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or levorotary) that they rotate the plane of polarized light at the wavelength of the sodium D line.

Some of the compounds of the present disclosure exist as ‘tautomeric isomers” or “tautomers.” “Tautomeric isomers” or “tautomers” are isomers that are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. Non-limiting examples of amide-comprising and imidic acid-comprising tautomers are shown below:

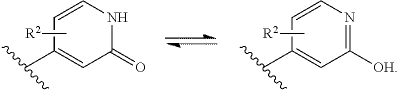

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to a complex formed by combining a compound and a solvent.

The term “hydrate” refers to the complex formed by combining a compound and water.

The term “pharmaceutically acceptable salt” of a given compound refers to salts that retain the biological effectiveness and properties of the given compound, and which are not biologically or otherwise undesirable. In many cases, the compounds of this disclosure are capable of forming pharmaceutically acceptable acid and/or base salts by virtue of the presence of amino and/or carboxyl groups or groups similar thereto.

Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts also may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms “pharmaceutically acceptable carrier” or “pharmaceutically acceptable excipient” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Any formula or structure given herein, including Formula (I) compounds, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present invention, for example those into which radioactive isotopes such as ³H, ¹³C, and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients.

Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An ¹⁸F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula (I).

The concentration of such a heavier isotope, specifically deuterium, may be defined by an isotopic enrichment factor. In the compounds of this invention any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this invention any atom specifically designated as a deuterium (D) is meant to represent deuterium.

In the description, including the examples, all temperatures are in degrees Celsius (° C.), unless otherwise stated, and abbreviations and acronyms have the following meanings:

| Abbreviation | Meaning |
|---|---|
| ° C. | Degree Celsius |
| 5-HIAA | 5-Hydroxyindoleacetic acid |
| 5-HIAL | 5-Hydroxyindoleacetaldehyde |
| 5-HT | 5-Hydroxytryptamine (serotonin) |
| 5-HTOL | 5-Hydroxytryptophol |
| Ae | Enzyme activities measured in the presence of a test compound |
| AIDS | Acquired immune deficiency syndrome |
| ALDH-2 | Human mitochondrial aldehyde dehydrogenase |
| Ao | Enzyme activities measured in the absence of a test compound |
| BHA | Butylated hydroxy anisole |
| BOC | tert-Butoxycarbonyl |
| BOP | Benzotriazolyl-N-hydroxytris(dimethyamino)phosphonium |
| | hexafluorophosphate |
| Cbz | Benzyl carbamate |
| cm | centimeter |
| d | Doublet |
| dd | Doublet of doublets |
| DA | Dopamine |
| DCC | Dicyclohexyl carbodiimide |
| DCM | Dichloromethane |
| DIC | Diisopropyl carbodiimide |
| DIEA | N,N-Diisopropylethylamine |
| DMF | Dimethylformamide |
| DMSO | Dimethylsulfoxide |
| dt | Doublet of triplets |
| EDTA | Ethylenediaminetetraacetic acid |
| equiv/eq | Equivalents |
| EtOAc | Ethyl acetate |
| EtOH | Ethanol |
| FR | Fixed ratio |
| g | Grams |
| HATU | O-(7-Azabenzotriazol-1-yl)-N,N,N′,N′-tetramethyluronium |
| | hexafluorophosphate |
| HBTU | O-Benzotriazole-N,N,N′,N′-tetramethyl-uronium- |
| | hexafluoro-phosphate |
| HPLC | High-performance liquid chromatography |
| hrs/h | Hours |
| Hz | Hertz |
| IC50 | The half maximal inhibitory concentration |
| IIDQ | 1-Isobutoxycarbonyl-2-isobutoxy-1,2-dihydro quinone |
| ip | Intraperitoneal |
| iv | Intravenous |
| J | Coupling constant |
| Kg | Kilogram |
| L | Liter |
| LAD | Low alcohol-drinking rat |
| LCMS/LC-MS | Liquid chromatography-mass spectrometry |
| LG | Leaving group |
| M | Molar |
| m/z | mass-to-charge ratio |
| M+ | Mass peak |
| M + H | Mass peak plus hydrogen |
| M + Na | Mass peak plus sodium |
| MAO | Monoamine oxidase |
| Me | Methyl |
| mg | Milligram |
| MHz | Megahertz |
| min | Minute |
| ml/mL | Milliliter |
| mM | Millimolar |
| mmol | Millimole |
| MOM | Methoxylmethyl |
| MS | Mass spectroscopy |
| NAD | Nicotinamide Adenine Dinucleotide |
| NaPPi | Sodium pyrophosphate |
| NIH | National Institute of Health |
| NMM | N-Methylmorpholine |
| NMR | Nuclear magnetic resonance |
| NP | Alcohol non-preferring rat |
| OCD | Obsessive compulsive disorder |
| PG | Protecting group |
| Ph | Phenyl |
| PyBOP | (Benzotriazol-1-yloxy)tripyrrolidinophosphonium hexafluorophosphate |
| q.s. | Quantity sufficient to achieve a stated function |
| RT/rt/R.T | Room temperature |
| s | Second |
| s | Singlet |
| SA | Self-administration |
| sc | Subcutaneous |
| SEM | Standard error of means |
| t | Triplet |
| TEA | Triethylamine |
| TES | Triethylsilyl |
| TFA | Trifluoroacetic acid |
| THF | Tetrahydrofuran |
| TIPS | Triisopropylsilyl |
| TKK | TKK buffer |
| TLC | Thin layer chromatography |
| TMS | Trimethylsilyl |
| TO | Time out |
| Tris | tris(hydroxymethyl)aminomethane |
| δ | Chemical shift |
| μg | Microgram |
| μL/μl | Microliter |
| μM | Micromolar |
| μmol | Micromole |

Dopamine-Producing Agents and Nicotine Addiction

Well-known dopamine-producing agents include alcohol, opioids, amphetamines, other drugs of addiction, foods (e.g., sugary foods), and nicotine. It is now well-established that dopamine-producing agents when administered to mammals (e.g., humans) induce surges in dopamine levels (either directly or indirectly) that can result in the acquisition of a conditioned response leading to the deleterious side-effect of addiction (e.g., misuse, dependence, abuse).

Nicotine exerts its dopamine-producing effect by binding to neuronal nicotinic acetylcholine receptors (nAChRs). Presynaptic nAChRs on midbrain dopamine neurons project from the ventral tegmental area to the nucleus accumbens and prefrontal cortex. These presynaptic nAChRs induce dopamine release when activated by nicotine. Sacco et al., “Nicotinic receptor mechanisms and cognition in normal states and neuropsychiatric disorders,” J. Psychopharmacol. 18:457-474 (2004).

Varenicline

Varenicline, as the tartrate salt, (7,8,9,10-tetrahydro-6,10-methano-6H-pyrazino[2,3-h][3]benzazepine, (2R,3R)-2,3-dihydroxybutanedioate(1:1)), is the active ingredient in CHANTIX™, one of the currently available therapies for nicotine addiction. It is contemplated that any pharmaceutically acceptable formulation of 7,8,9,10-tetrahydro-6,10-methano-6H-pyrazino[2,3-h][3]benzazepine can be used in the compositions and methods of the present disclosure. Such formulations include but are not limited to a single stereoisomer, a mixture of stereoisomers, one or more tautomers, or pharmaceutically acceptable salts other than tartrate. The preparation of varenicline, its pharmaceutical compositions, formulations, and methods for their use in treatment are disclosed in e.g., U.S. Pat. Nos. 6,410,550, 6,890,927, and 7,265,119, each which is hereby incorporated by reference herein. Some of these U.S. patents also refer to varenicline in its free base form by the chemical name: 5,8,14-triazatetracyclo[10.3.1.0^{2,11}.0^{4,9}]-hexadeca-2(11),3,5,7,9-pentaene.

Varenicline is not an ALDH-2 inhibitor. Rather it is a partial agonist of α4β2 neuronal nicotinic acetylcholine receptors and stimulates receptor-mediated activity, but at a level significantly lower than nicotine-induced stimulation. Additionally, varenicline binding to α4β2 receptors inhibits nicotine-induced stimulation of the central nervous mesolimbic dopamine system that contributes the reward experienced upon smoking.

The FDA label for CHANTIX™ approves it for the treatment of nicotine addiction and/or as an aid in the cessation of tobacco use in humans. The currently FDA-approved mode for administration of varenicline to adults is orally as tablets containing 0.85 mg or 1.71 mg of varenicline tartrate salt, which is equivalent to 0.50 mg or 1.00 mg of varenicline. The recommended dose of CHANTIX™ is 1.0 mg administered twice daily following a 1-week titration administered according to the following schedule. Days 1-3: 0.5 mg once daily; days 4-7: 0.5 mg twice daily; day 8-end of treatment: 1.0 mg twice daily.

Common side-effects identified as associated with the use of the approved dosages of varenicline to aid in smoking cessation include mild nausea (˜30% of patients), headaches, difficulty sleeping, and nightmares. The incidence of nausea was found to be higher at larger doses (30%) versus placebo (10%) relative to incidence at smaller doses (16%) versus placebo (11%). Gastrointestinal side-effects have been estimated lead to varenicline discontinuation in 2%-8% of patients.

In view of it is side-effects, the present disclosure provides methods and pharmaceutical compositions that result in an amount of varenicline administered in combination with an ALDH-2 inhibitor that is significantly lower than the recommended effective amount of varenicline when it is administered alone. Accordingly, the present disclosure provides for the therapeutically effective amount of varenicline used in combination with an ALDH-2 inhibitor (e.g., compound (2) disclosed herein) comprising an amount of varenicline that can be (a) less than 2.0 mg, less than 1.0 mg, less than 0.5 mg, less than 0.25 mg, less than 0.1 mg, or less than 0.05 mg; (b) between about 0.05 mg and 2.0 mg, between about 0.05 mg and 1.0 mg, between about 0.05 mg and 0.5 mg, between about 0.05 mg and 0.4 mg, between about 0.05 mg and 0.25 mg, or between about 0.05 mg and 0.15 mg; or (c) less than 2.0 mg/day, less than 1.0 mg/day, less than 0.5 mg/day, less than 0.25 mg/day, or less than 0.1 mg/day.

ALDH-2 Inhibitor Compounds

Compounds that act as selective inhibitors of ALDH-2 have been shown to reduce pathophysiologic dopamine surge without changing basal dopamine levels. See e.g., Yao et al., “Inhibition of aldehyde dehydrogenase-2 suppresses cocaine seeking by generating THP, a cocaine use-dependent inhibitor of dopamine synthesis,” Nature Medicine (2010), Vol. 16, No. 9; Diamond and Yao, “From Ancient Chinese Medicine to a Novel Approach to Treat Cocaine Addiction,” CNS & Neurological Disorders—Drug Targets (2015) Vol. 14, No. 6. Selective inhibitors of ALDH-2 have also demonstrated the ability to suppress self-administration of nicotine in rats. See e.g., Rezvani et al., “Inhibition of Aldehyde Dehydrogenase-2 (ALDH-2) Suppresses Nicotine Self-Administration in Rats,” (2015) Journal of Drug and Alcohol Research, vol. 4: 1-6; and U.S. Pat. Nos. 8,558,001, 8,575,353, 9,000,015, 9,610,299; Int'l Pat. Publ. WO2013/006400.

The ALDH-2 inhibitor compounds, compositions, and methods provided in the present disclosure have been shown to be useful for the reduction and/or prevention of addiction in mammals to nicotine, and other dopamine-producing agents. ALDH-2 inhibitor compounds useful in the methods and compositions of the present disclosure can include any of the compounds well-known in the art as ALDH-2 inhibitors including, but not limited to, daidzein (compound (15)), or its pharmaceutically acceptable salts, esters, or a tautomer thereof.

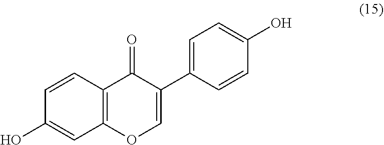 [SMILES: O=c1c(-c2ccc(O)cc2)coc2cc(O)ccc12]

ALDH-2 inhibitor compounds useful in the methods and compositions of the present disclosure can include the isoflavone compounds structurally related to daidzein, such as 3-{[3-(4-aminophenyl)-4-oxochromen-7-yloxy]methyl}benzoic acid (compound (16)), or its pharmaceutically acceptable salts, esters, or a tautomer thereof.

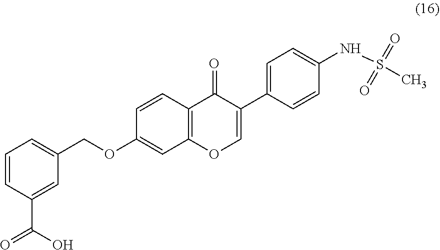 [SMILES: CS(=O)(=O)Nc1ccc(-c2coc3cc(OCc4cccc(C(=O)O)c4)ccc3c2=O)cc1]

Additional ALDH-2 inhibitor compounds comprising an isoflavone structure that are useful in the methods and compositions of the present disclosure are described in U.S. Pat. Nos. 5,624,910, 6,121,010 7,951,813, 8,158,810, and 8,673,966, and Int'l Pat. Publ. Nos. WO2008/014497, WO2008/124532, WO2009/061924, WO2009/094028, and WO2013/033377, each of which is hereby incorporated by reference herein.

ALDH-2 inhibitor compounds useful in the methods and compositions of the present disclosure can include any of the ALDH-2 inhibitor compounds that are structurally unrelated to daidzein and the other isoflavones. These include the ALDH-2 inhibitor compounds described in U.S. Pat. Nos. 8,558,001, 8,575,353, 9,000,015, 9,610,299, Intl Pat. Publ. WO2013/006400, each of which is hereby incorporated by reference herein. Accordingly, in some embodiments of the methods and compositions of the present disclosure, the ALDH-2 inhibitor compound used is a compound of Formula (I):

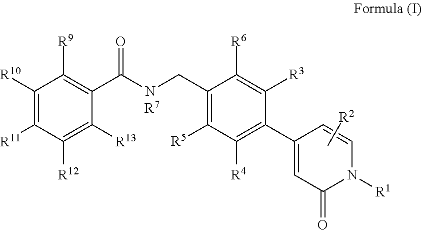

wherein:

R¹ is hydrogen, optionally substituted C₁₋₆ alkyl, —CH₂OH, —CH₂OP(O)(OR²⁰)(OR²¹);

R² is hydrogen, optionally substituted C₁₋₆ alkyl, cycloalkyl, or halo;
each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O—(C₁ to C₆-alkyl)-O—(C₁ to C₆-alkyl), cyano, halo, —SO₂NR²⁴R²⁵; or —NR²⁴R²⁵;

R⁷ is hydrogen or optionally substituted C₁₋₆ alkyl;
each of R²⁰ and R²¹ is independently Na⁺, Li⁺, K⁺, hydrogen, C₁₋₆ alkyl; or R²⁰ and R²¹ can be combined to represent a single divalent cation Zn²⁺, Ca²⁺, or Mg²⁺; and

each of R²⁴ and R²⁵ is independently chosen from hydrogen or C₁₋₆ alkyl or when combined together with the nitrogen to which they are attached form a heterocycle; or

a pharmaceutically acceptable salt, ester, single stereoisomer, mixture of stereoisomers, or a tautomer thereof.

The naming and numbering of the compounds of Formula (I) is illustrated with a representative compound (1):

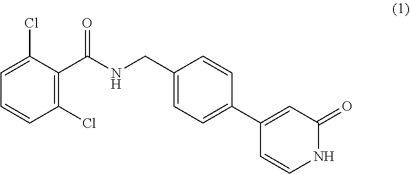 [SMILES: O=C(NCc1ccc(-c2cc[nH]c(=O)c2)cc1)c1c(Cl)cccc1Cl]

namely: 2,6-dichloro-N-[4-(2-oxo-1,2-dihydro-pyridin-4-yl)-benzyl]-benzamide.
In certain embodiments, R¹ is hydrogen. In certain embodiments, R¹ is C₁₋₆ alkyl. In certain embodiments, R¹ is methyl. In certain embodiments, R¹ is —CH₂OP(O)(OR²⁰)(OR²¹); and each of R²⁰ and R²¹ is independently Na⁺, Li⁺, K⁺, or hydrogen. In certain embodiments, at least one of R¹, R⁹, R¹⁰, R¹¹, R¹², R¹³, is not hydrogen. In other embodiments, at least two of R¹, R⁹, R¹⁰, R¹¹, R¹², R¹³ is not hydrogen.

In certain embodiments, R² is hydrogen. In certain embodiments, R² is C₁₋₆ alkyl. In certain embodiments, R² is methyl. In certain embodiments, R² is selected from the group consisting of ethyl, n-propyl, iso-propyl, n-butyl, iso-butyl, t-butyl, and n-hexyl. In certain embodiments, R² is halo. In certain embodiments, R² is fluoro. In certain embodiments, R² is chloro. In certain embodiments, R² is bromo. In certain embodiments, R² is iodo.

In certain embodiments, each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted C₁₋₆ alkyl, optionally substituted C₃₋₈ cycloalkyl, optionally substituted C₁₋₆ alkoxy, —O—(C₁ to C₆-alkyl)-O—(C₁ to C₆-alkyl), —C(O)NH₂, cyano, or halo. In certain embodiments, each of R³, R⁴, R⁵, and R⁶ is independently hydrogen, C₁₋₆ alkyl, or halo. In certain embodiments, one of R³, R⁴, R⁵, or R⁶ is C₁₋₆ alkyl or halo. In certain embodiments, one of R³, R⁴, R⁵, or R⁶ is selected from the group consisting of ethyl, n-propyl, iso-propyl, n-butyl, iso-butyl, t-butyl, and n-hexyl. In certain embodiments, one of R³, R⁴, R⁵, or R⁶ is methyl. In certain embodiments, one of R³, R⁴, R⁵, or R⁶ is fluoro. In certain embodiments, one of R³, R⁴, R⁵, or R⁶ is chloro. In certain embodiments, one of R³, R⁴, R⁵, or R⁶ is fluoro. In certain embodiments, one of R³, R⁴, R⁵, or R⁶ is iodo.

In certain embodiments, R⁷ is hydrogen. In certain embodiments, R⁷ is C₁₋₆ alkyl. In certain embodiments, R⁷ is selected from the group consisting of ethyl, n-propyl, iso-propyl, n-butyl, iso-butyl, t-butyl, and n-hexyl. In certain embodiments, R⁷ is methyl.

In certain embodiments, at least one of R⁹ and R¹³ is not hydrogen. In certain embodiments, at least one of R⁹ and R¹³ is halo or C₁₋₆ alkyl. In certain embodiments, at least one of R⁹ and R¹³ is selected from the group consisting of ethyl, n-propyl, iso-propyl, n-butyl, iso-butyl, t-butyl, and n-hexyl. In certain embodiments, at least one of R⁹ and R¹³ is independently chloro, fluoro, or methyl. In certain embodiments, at least one of R⁹ and R¹³ is bromo. In certain embodiments, at least one of R⁹ and R¹³ is iodo. In certain embodiments, R⁹ and R¹³ are independently halo or C₁₋₆ alkyl. In certain embodiments, R⁹ and R¹³ are independently chloro, fluoro, or methyl. In certain embodiments, R⁹ and R¹³ are chloro. In certain embodiments, R⁹ and R¹³ are methyl.

In certain embodiments, each of R¹⁰ and R¹² is independently hydrogen, halo, or C₁₋₆ alkyl. In certain embodiments, each of R¹⁰ and R¹² is independently ethyl, n-propyl, iso-propyl, n-butyl, iso-butyl, t-butyl, and n-hexyl. In certain embodiments, each of R¹⁰ and R¹² is independently hydrogen, chloro, fluoro, or methyl. In certain embodiments, each of R¹⁰ and R¹² is independently bromo. In certain embodiments, each of R¹⁰ and R¹² is independently iodo. In certain embodiments, each of R¹⁰ and R¹² is independently fluoro. In certain embodiments, each of R¹⁰ and R¹² is independently chloro. In certain embodiments, R¹⁰ and R¹² are hydrogen.

In certain embodiments, R¹¹ is hydrogen. In certain embodiments, R¹¹ is —O—(C₁ to C₆-alkyl)-O—(C₁ to C₆-alkyl). In certain embodiments, R^{H} is —OCH₂CH₂OCH₃. In certain embodiments, R¹¹ is independently ethyl, n-propyl, iso-propyl, n-butyl, iso-butyl, t-butyl, and n-hexyl. In certain embodiments, R¹¹ is halo. In certain embodiments, R¹¹ is fluoro. In certain embodiments, R¹¹ is chloro. In certain embodiments, R¹¹ is bromo. In certain embodiments, R¹¹ is iodo.

In certain embodiments,

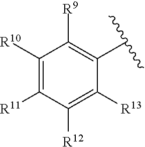

is selected from the group consisting of:

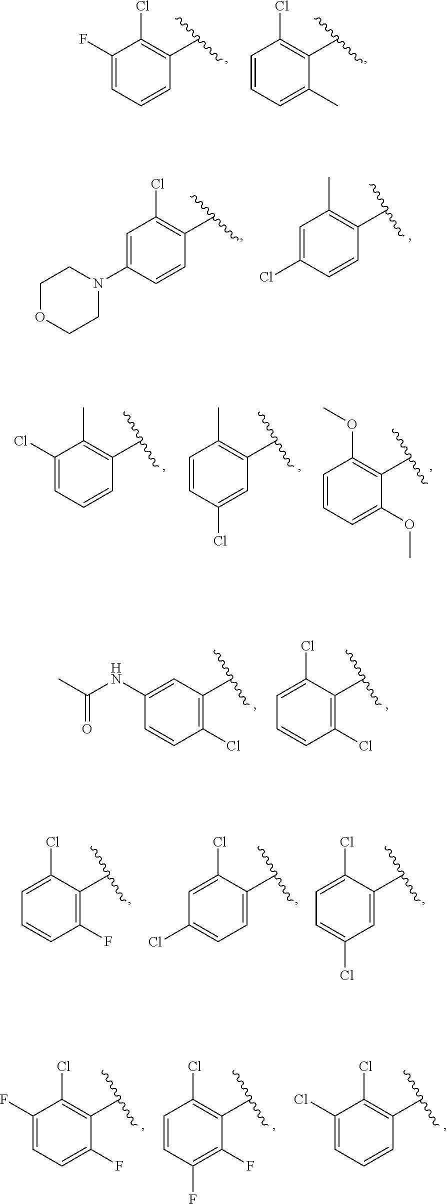

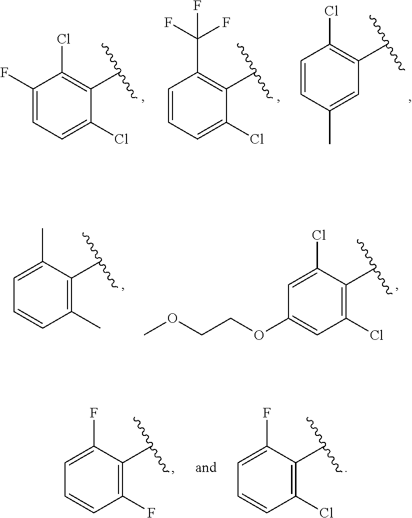

In certain embodiments, R¹ is hydrogen, methyl, or —CH₂OP(O)(OR²⁰)(OR²¹); R² is hydrogen, methyl, or fluoro; each of R³ and R⁴ is independently hydrogen or methyl; each of R⁵ and R⁶ is independently hydrogen or fluoro; R⁷ is hydrogen; R⁹ is hydrogen, chloro, fluoro, or methyl; R¹⁰ is hydrogen or fluoro; R¹¹ is hydrogen or —OCH₂CH₂OCH₃; R¹² is hydrogen or fluoro; R¹³ is hydrogen, chloro, fluoro, or methyl; and each of R²⁰ and R²¹ is independently Na⁺, Li⁺, K⁺, or hydrogen.

In certain embodiments, the ALDH-2 inhibitor compound of Formula (I) is selected from the group consisting of the compounds (1)-(14) listed in Table 1. As described in U.S. Pat. No. 8,558,001, each of these compounds exhibits high, selective inhibition of the human ALDH-2 enzyme, with IC₅₀ values of less than 1 μm, and relatively low inhibitory activity toward the MAO-A and MAO-B pathway enzymes, with IC₅₀ values of >130 μm. It should be noted that high IC₅₀ value for compound (2) is due to it being a phosphoric acid adduct prodrug of compound (1). Thus, compound (2) undergoes in vivo cleavage of the phosphoric acid group to yield compound (1).

TABLE 1
Exemplary ALDH-2 Inhibitor Compounds of Formula (I)

| | | IC₅₀ | IC₅₀ | IC₅₀ |
|---|---|---|---|---|
| Compound | | ALDH-2 | hMAO-A | hMAO-B |
| No. | Compound Name | (nm) | (μm) | (μm) |
| (1) | 2,6-dichloro-N-[4-(2-oxo-1,2-dihydro-pyridin-4-yl)-benzyl]-benzamide | 102 | >130 | >130 |
| (2) | phosphoric acid mono-(4-{4-[(2,6-dichloro-benzoylamino)-methyl]-phenyl}-2-oxo-2H-pyridin- | >10000.00 | >129.51 | >130 |
| | 1-ylmethyl) ester | | | |
| (3) | 2,6-dichloro-4-(2-methoxyethoxy)-N-(4-(2-oxo-1,2-dihydropyridin-4-yl)benzyl)benzamide | 63 | >130 | >130 |
| (4) | 2-chloro-3-fluoro-N-(4-(2-oxo-1,2-dihydropyridin-4-yl)benzyl)benzamide | 215 | >130 | >130 |
| (5) | 2-chloro-6-methyl-N-(4-(2-oxo-1,2-dihydropyridin-4-yl)benzyl)benzamide | 23 | >130 | >130 |
| (6) | 2,6-dimethyl-N-(4-(2-oxo-1,2-dihydropyridin-4-yl)benzyl)benzamide | 166 | >130 | >130 |
| (7) | 2,6-dichloro-N-[4-(6-methyl-2-oxo-1,2-dihydro-pyridin-4-yl)-benzyl]-benzamide | 1113 | >130 | >130 |
| (8) | 2-chloro-3,6-difluoro-N-(4-(2-oxo-1,2-dihydropyridin-4-yl)benzyl)benzamide | 464 | >130 | >130 |
| (9) | 2,6-dichloro-N-(3-methyl-4-(2-oxo-1,2-dihydropyridin-4-yl)benzyl)benzamide | 480 | >130 | >130 |
| (10) | 2,6-dichloro-N-(4-(1-methyl-2-oxo-1,2-dihydropyridin-4-yl)benzyl)benzamide | 2093 | >130 | >130 |
| (11) | 2,6-difluoro-N-(4-(2-oxo-1,2-dihydropyridin-4-yl)benzyl)benzamide | 890 | >130 | >130 |
| (12) | 2-chloro-6-fluoro-N-(4-(2-oxo-1,2-dihydropyridin-4-yl)benzyl)benzamide | 379 | >130 | >130 |
| (13) | 2,6-dichloro-N-(2-fluoro-4-(2-oxo-1,2-dihydropyridin-4-yl)benzyl)benzamide | 304 | >130 | >130 |
| (14) | 2,6-dichloro-N-(4-(5-fluoro-2-oxo-1,2-dihydropyridin-4-yl)benzyl)benzamide | 25 | >130 | >130 |

In certain embodiments, the compound of Formula (I) is compound (1):

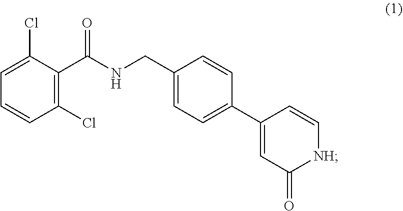 [SMILES: O=C(NCc1ccc(-c2cc[nH]c(=O)c2)cc1)c1c(Cl)cccc1Cl]

or a pharmaceutically acceptable salt, ester, single stereoisomer, mixture of stereoisomers, or tautomer thereof.

In certain embodiments, the compound of Formula (I) is compound (2):

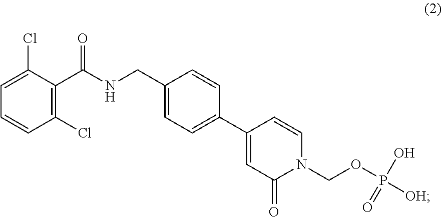 [SMILES: O=C(NCc1ccc(-c2ccn(COP(=O)(O)O)c(=O)c2)cc1)c1c(Cl)cccc1Cl]

or a pharmaceutically acceptable salt, ester, single stereoisomer, mixture of stereoisomers, or tautomer thereof. As noted above, compound (2) is an exemplary prodrug compound of Formula (I). It loses generates the free amide (pyridine) compound (1) in vivo as a metabolite. Accordingly, one of ordinary skill in the art can synthesize other prodrugs of compounds of Formula (I) based on the disclosure herein and synthetic methods well-known in the art.

Preparation of ALDH-2 Inhibitor Compounds of Formula (I)

The compounds of Formula (I) can be prepared from readily available starting materials using methods and procedures known in the art. In particular, the disclosure of U.S. Pat. No. 8,558,001 (Cannizzaro et al.) issued Oct. 15, 2013, which is hereby incorporated by reference herein, provides general synthetic strategies for preparing compounds of Formula (I), and also exemplifies specific synthesis protocols that can be used to prepare the compounds (1), (2), (3), (4), (5), (6), (7), (8), (9), (10), (11), (12), (13), and (14) described herein and listed above in Table 1. Further, the synthetic protocol for the preparation of compounds (1) and (2) is provided below in the Examples of the present disclosure.

Briefly, the compounds of Formula (I) may be prepared according to the synthetic sequence shown in Scheme I

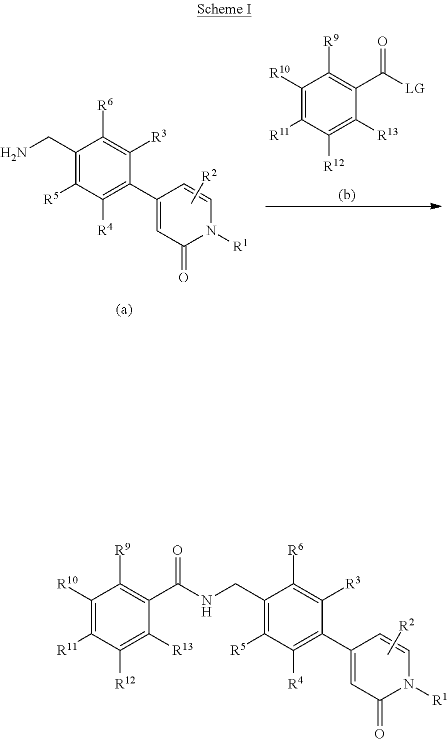

wherein, substituents R¹ through R²⁷, X¹, Y¹, Z¹ and Z² are as defined herein; LG is a leaving group (e.g., halo, hydroxyl, alkoxy, OSO₂CF₃, N₂⁺, etc.); PG is a protecting group (e.g., t-butyl, t-butyl carbamate (BOC), etc.); and Z² is (OH)₂, (OMe)₂, F³⁻, or (OR^{H})(OR^{J}), wherein OR^{H} and OR^{J} may combine with boron to form a cyclic arylboronic ester moiety or cyclic alkylboronic ester moiety as described herein (e.g., 4,4,5,5-tetramethyl-1,3,2-dioxaboronic ester, catechol dioxaboronic ester, etc.); wherein R17 is an optionally substituted alkylene moiety of 1-6 carbon atoms.

The Scheme I reactants (a) and (b) are commercially available or can be prepared by means well known in the art. In general, the reactants (a) and at least one molar equivalent, and preferably a slight excess (e.g., 1.2 to 1.5 molar equivalents) of (b), as shown in Scheme I, are combined under standard reaction conditions in an inert solvent, such as dimethylformamide (DMF), at a temperature of about 25° C. until the reaction is complete, generally about 16 hours. Standard reaction conditions may comprise the use of a molar excess of suitable base, such as sodium or potassium hydroxide, triethylamine, diisopropylethylamine, N-methylmorpholine (NMM), or pyridine, or in some cases where LG is hydroxyl, a peptide coupling reagent, such as O-(7-azabenzotriazol-1-yl)-N,N,N′,N′-tetra methyluronium hexafluorophosphate (HATU), may be used. When the reaction is substantially complete, the product is subjected, if necessary, to a deprotection sequence under standard reaction conditions (e.g., THF, CH₂Cl₂, or the like, a molar excess of acid such as acetic acid, formic acid, trifluoroacetic acid, or the like as described herein) to yield isolated by conventional means. Further alternative synthetic methods for preparing compounds of Formula (I) are described in the synthetic sequences of Schemes II-V as disclosed in U.S. Pat. No. 8,558,001.

It will be appreciated that where typical or preferred process conditions (i.e., reaction temperatures, times, mole ratios of reactants, solvents, pressures, etc.) are disclosed in U.S. Pat. No. 8,558,001, other process conditions can also be used unless otherwise stated. Optimum reaction conditions may vary with the particular reactants or solvent used, but such conditions can be determined by one skilled in the art by routine optimization procedures. Additionally, as will be apparent to those skilled in the art, conventional protecting groups may be necessary to prevent certain functional groups from undergoing undesired reactions. The term “protecting group” or “PG,” as used herein, is meant that a particular functional moiety, e.g., O, S, or N, is temporarily blocked so that a reaction can be carried out selectively at another reactive site in a multifunctional compound. “Protecting groups” or “PGs,” as used herein, are well known in the art and include those described in detail in Protective Groups in Organic Synthesis, Fourth Ed., Greene, T. W. and Wuts, P. G., Eds., John Wiley & Sons, New York: 2007, the entire contents of which are hereby incorporated by reference, and references cited therein.

The starting materials for the synthetic reactions Schemes I-V as disclosed in U.S. Pat. No. 8,558,001 are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemie or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

Methods of Use

The present disclosure provides methods of use and treatment comprising administering to said mammal a therapeutically effective amount of varenicline in combination with a therapeutically effective amount of an ALDH-2 inhibitor (e.g., compound of Formula (I)). These methods of treatment act to reduce nicotine addiction and/or aid in the cessation or lessening of tobacco use in a mammal. While not wishing to be bound by theory, ALDH-2 inhibitors (such as the compounds of Formula (I)) are known to be effective in reducing or preventing surges in dopamine levels caused by administration of a substance containing a dopamine-producing agent. It is believed that, as a consequence of this ability of ALDH-2 inhibitors to reduce surges in dopamine, they also can reduce or prevent an addiction to nicotine, and/or aid in the cessation of tobacco use. Based on this proposed mechanism of action, the ALDH-2 inhibitors (such as the compounds of Formula (I)) can be administered in combination with substances that treat nicotine addiction by other mechanisms of action, such as varenicline (which, as described above, acts as a partial agonist of the α4β2 nicotinic acetylcholine receptor subtypes), and thereby reduce or prevent nicotine addiction in a patient receiving the combination treatment.

Accordingly, the methods of the present disclosure comprise administering to a mammal in need thereof a therapeutically effective dose of an ALDH-2 inhibitor in combination with varenicline. The two active ingredients (ALDH-2 inhibitor and varenicline) can be administered in combination with each other either separately or together (e.g., simultaneously). If administered separately, however, it is contemplated that the ALDH-2 inhibitor compound and varenicline be administered close enough in time such that levels of the ALDH-2 inhibitor present in the subject are sufficient to provide for the synergistic effect associated with the co-administration of the varenicline.

In some embodiments of the method, the administration in combination comprises administering the therapeutically effective dose of the ALDH-2 inhibitor prior to administration of the therapeutically effective dose of the varenicline. In some embodiments, it is contemplated that the ALDH-2 is administered as a once-a-day dose. In some embodiments, the once-a-day dose is in a formulation (e.g., a tablet), that is self-administered by the subject or patient.

Additionally, it is contemplated in some embodiments of the methods, that the administration in combination comprises administering a therapeutically effective dose of the ALDH-2 inhibitor once-a-day and administering a therapeutically effective dose of varenicline at least two or more times a day.

In some embodiment of the methods, the administration in combination comprises administering the therapeutically effective dose of the ALDH-2 inhibitor simultaneously with administration of the therapeutically effective dose of varenicline. For example, it is contemplated that a patient in thereof could self-administer an oral dosage form of the ALDH-2 inhibitor and an oral dosage form of varenicline simultaneously, e.g., two tablets taken at the same time.

In some embodiments of the methods, it is contemplated that the administration in combination comprises administering a pharmaceutical composition comprising both the therapeutically effective dose of the varenicline, and the therapeutically effective dose of the ALDH-2 inhibitor compound, as well as a pharmaceutically acceptable carrier. In some embodiments, it is contemplated that this pharmaceutical composition comprising the two active ingredients of the varenicline and the ALDH-2 inhibitor is formulated in a combination dosage form. Thus, in some embodiments of the method the administration in combination can comprise self-administration of a combination dosage form, e.g., a single tablet, that comprises both active ingredients of the combination. Such embodiments include methods wherein the varenicline and the ALDH-2 inhibitor are administered as combination dosage form, optionally, an oral combination dosage form.

As contemplated by the present disclosure, the different mechanisms of action of varenicline and a selective ALDH-2 inhibitor (e.g., compound (2)) can provide a synergistic effect in reducing nicotine addiction and/or aiding in cessation of tobacco use. It is contemplated that the methods of present disclosure comprise administering a therapeutically effective amount of varenicline that is less than the amount provided in the FDA approved forms of varenicline (CHANTIX™). The recommended dose of CHANTIX™ is 1.0 mg administered twice daily following a 1-week titration administered according to the following schedule. Days 1-3: 0.5 mg once daily; days 4-7: 0.5 mg twice daily; day 8-end of treatment: 1.0 mg twice daily. Accordingly, in some embodiments, the methods of the present disclosure comprise a therapeutically effective dose of varenicline (when used in combination with an ALDH-2 inhibitor) that is (a) less than 2.0 mg, less than 1.0 mg, less than 0.5 mg, less than 0.25 mg, less than 0.1 mg, or less than 0.05 mg; (b) between about 0.05 mg and 2.0 mg, between about 0.05 mg and 1.0 mg, between about 0.05 mg and 0.5 mg, between about 0.05 mg and 0.4 mg, between about 0.05 mg and 0.25 mg, or between about 0.05 mg and 0.15 mg; or (c) less than 2.0 mg/day, less than 1.0 mg/day, less than 0.5 mg/day, less than 0.25 mg/day, or less than 0.1 mg/day.

Similarly, it is contemplated that the methods of present disclosure comprise administering a therapeutically effective amount of the ALDH-2 inhibitor that is less than the amount determined effective in published pre-clinical animal studies. See e.g., Rezvani et al., “Inhibition of Aldehyde Dehydrogenase-2 (ALDH-2) Suppresses Nicotine Self-Administration in Rats,” (2015) Journal of Drug and Alcohol Research, vol. 4: 1-6; and U.S. Pat. Nos. 8,558,001, 8,575,353, 9,000,015, 9,610,299; Int'l Pat. Publ. WO2013/006400. Accordingly, in some embodiments, the methods of the present disclosure comprise a therapeutically effective dose of ALDH-2 inhibitor (when administered in combination with varenicline) that is: (a) less than 5 mg, less than 2.5 mg, less than 1.0 mg, or less than 0.5 mg; (b) between 0.5 mg and 5 mg, between about 0.5 mg and 4.0 mg, between about 0.5 mg and 2.5 mg, or between about 0.5 mg and 1.5 mg; or (c) less than 5 mg/day, less than 2.5 mg/day, or less than 1.0 mg/day of the ALDH-2 inhibitor.

As noted above, in some embodiments of the methods the varenicline and the ALDH-2 inhibitor can be formulated as, and administered in, a combination dosage form (e.g., a pharmaceutical composition of varenicline, the ALDH-2 inhibitor, and a pharmaceutically acceptable carrier). It is contemplated that due to the synergistic effect of combining varenicline and an ALDH-2 inhibitor, the combination dosage form will comprise therapeutically effective doses that are lower than previously considered when using each compound alone to treat nicotine addiction. Accordingly, in some embodiments, in the methods of the present disclosure the therapeutically effective doses of varenicline and the ALDH-2 inhibitor when administered in combination can comprise: (a) less than 0.5 mg, less than 0.25 mg, less than 0.1 mg, or less than 0.05 mg of varenicline, and less than 5 mg, less than 2.5 mg, less than 1.0 mg, or less than 0.5 mg of the ALDH-2 inhibitor; (b) between 0.05 mg and 0.5 mg, between about 0.05 mg and 0.4 mg, between about 0.05 mg and 0.25 mg, or between about 0.05 mg and 0.15 mg of varenicline, and between 0.5 mg and 5 mg, between about 0.5 mg and 4.0 mg, between about 0.5 mg and 2.5 mg, or between about 0.5 mg and 1.5 mg of the ALDH-2 inhibitor; or (c) less than 0.5 mg/day, less than 0.25 mg/day, or less than 0.1 mg/day of varenicline, and less than 5 mg/day, less than 2.5 mg/day, or less than 1.0 mg/day of the ALDH-2 inhibitor.

Generally, it is contemplated that the methods of combination treatment disclosed herein can be used with any dopamine-producing agent associated with addiction for which a course of treatment with varenicline is indicated.

As described above, in some embodiments of the methods, the mammal (e.g., human patient) self-administers the pharmaceutically effective amount of the varenicline in combination with the pharmaceutically effective amount of the ALDH-2 inhibitor. Accordingly, in another aspect the present disclosure provides a patient pack comprising at least one pharmaceutical composition that comprises at least one of the active ingredients as described herein (e.g., a pharmaceutical composition comprising the varenicline and/or the ALDH-2 inhibitor) and an information package or product insert containing directions on the method of using the pharmaceutical compositions.

Accordingly, in some embodiments, the present disclosure provides a method of reducing or preventing in a mammal the addiction to nicotine, wherein the method comprises administering to the mammal a therapeutically effective amount of an ALDH-2 inhibitor in combination with varenicline; optionally, wherein the ALDH-2 inhibitor is a compound of Formula (I).

In the various embodiments of the methods disclosed herein, the step of administering the varenicline in combination with the ALDH-2 inhibitor can comprise administering a pharmaceutical composition, wherein the pharmaceutical composition comprises the varenicline, the ALDH-2 inhibitor, and a pharmaceutically acceptable carrier.

Pharmaceutical Compositions

In some embodiments of the methods of the present disclosure, it is contemplated that the varenicline and the ALDH-2 inhibitor are administered in combination with each other in the form of pharmaceutical compositions. When administered in separate doses, each dosage contains a therapeutically effective amount of the active ingredient (i.e., the varenicline or the ALDH-2 inhibitor), or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants.

As noted above, in some embodiments of the methods of the present disclosure, the step of administering the varenicline in combination with an ALDH-2 inhibitor can comprise administering a pharmaceutical composition, wherein the pharmaceutical composition is a combination composition that contains the varenicline, the ALDH-2 inhibitor (e.g., compound (2)), and a pharmaceutically acceptable carrier. Accordingly, in some embodiments the present disclosure also provides a pharmaceutical composition, wherein the composition comprises a therapeutically effective amount of varenicline, a therapeutically effective amount of an ALDH-2 inhibitor, and a pharmaceutically acceptable carrier. In some embodiments, the combination pharmaceutical composition is in a unit dosage form, such as a combination dosage form that contains a combination of the active ingredients (e.g., ALDH-2 inhibitor and varenicline) in a single dosage form.

In some embodiments, the present disclosure provides a combination dosage form comprising a pharmaceutical composition of varenicline, the ALDH-2 inhibitor, and a pharmaceutically acceptable carrier, wherein the combination dosage form comprises:

(a) less than 2.0 mg, less than 1.0 mg, less than 0.5 mg, less than 0.25 mg, less than 0.1 mg, or less than 0.05 mg of varenicline, and less than 5 mg, less than 2.5 mg, less than 1.0 mg, or less than 0.5 mg of the ALDH-2 inhibitor;

(b) less than 2.0 mg, less than 1.0 mg, between about 0.05 mg and 0.5 mg, between about 0.05 mg and 0.4 mg, between about 0.05 mg and 0.25 mg, or between about 0.05 mg and 0.15 mg of varenicline, and between 0.5 mg and 5 mg, between about 0.5 mg and 4.0 mg, between about 0.5 mg and 2.5 mg, or between about 0.5 mg and 1.5 mg of the ALDH-2 inhibitor; or

(c) less than 2.0 mg/day, less than 1.0 mg/day, 0.5 mg/day, less than 0.25 mg/day, or less than 0.1 mg/day of varenicline, and less than 5 mg/day, less than 2.5 mg/day, or less than 1.0 mg/day of the ALDH-2 inhibitor.

Such pharmaceutical compositions can be prepared using methods well known in the pharmaceutical art (see, e.g., Remington's Pharmaceutical Sciences, Mace Publishing Co., Philadelphia, Pa. 17th Ed. (1985) and Modern Pharmaceutics, Marcel Dekker, Inc. 3rd Ed. (G. S. Banker & C. T. Rhodes, Eds.). Methods of preparing pharmaceutical compositions of ALDH-2 inhibitor compounds, such as compounds of Formula (I), are described in e.g., U.S. Pat. Nos. 7,951,813, 8,158,810, 8,673,966, 8,558,001, 8,575,353, 9,000,015, and 9,610,299, each of which is hereby incorporated by reference herein. Methods for the preparation and use of pharmaceutical compositions comprising varenicline are also well-known in the art, and described in e.g., U.S. Pat. Nos. 6,410,550, 6,890,927, 7,265,119, and 8,314,235, each which is hereby incorporated by reference herein.

Administering the Pharmaceutical Compositions

In the methods of the present disclosure it is contemplated that the pharmaceutical composition(s) comprising the varenicline and the ALDH-2 inhibitor, such as a compound of Formula (I), can be administered in combination with each other, either as single or multiple doses, and by any of the accepted modes of administration of active ingredients having similar utility. For example, as described in U.S. Pat. No. 8,558,001, a pharmaceutical composition comprising an ALDH-2 inhibitor compound of Formula (I) can be administered using a variety of different modes including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

Varenicline, as sold by Pfizer under the brand name CHANTIX™ to aid adults in smoking cessation, is orally administered as tablets containing 0.85 mg or 1.71 mg of varenicline tartrate equivalent to 0.5 mg or 1 mg of varenicline. It is contemplated, however, that varenicline can be administered via modes other than oral administration including, but not limited to, via transdermal (e.g., through the use of a patch), intranasal, sublingual, rectal, parenteral or topical administration routes, as described n e.g., U.S. Pat. Nos. 6,890,927 and 7,265,119.

One exemplary route for administering that is useful in the methods of the present disclosure is oral. Oral administration may be via capsule, enteric coated tablets, or the like. Typically, in making the pharmaceutical compositions that include a medication containing a dopamine-producing agent and/or an ALDH-2 inhibitor, such as compound of Formula (I), the active ingredient(s) is diluted by an excipient and/or enclosed within a carrier in the form of a capsule, sachet, paper or other container. When the excipient serves as a diluent, it can be a solid, semi-solid, or liquid material (as above), which acts as a vehicle, carrier or medium for the active ingredient. Thus, the pharmaceutical composition(s) suitable for administering in the methods of the disclosure can be in the dosage form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Exemplary suitable excipients for the compositions of the present disclosure are well known in the art and include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical compositions can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The pharmaceutical compositions useful in the methods of the present disclosure can be formulated so as to provide quick, sustained or delayed release of the relevant active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in e.g., U.S. Pat. Nos. 3,845,770; 4,326,525; 4,902,514; and 5,616,345.

The pharmaceutical compositions useful in the methods of the present disclosure can also be formulated for administration via transdermal delivery devices (e.g., “patches”). Such transdermal patches may be used to provide continuous or discontinuous infusion of the pharmaceutical compositions in controlled amounts. The construction and use of transdermal patches for the delivery of pharmaceutical compositions is well known in the art. See, e.g., U.S. Pat. Nos. 5,023,252, 4,992,445 and 5,001,139. Such patches may be constructed for continuous, pulsatile, or on demand delivery of the pharmaceutical composition(s).

In some embodiments, the pharmaceutical composition(s) useful in the methods of the present disclosure are formulated in a unit dosage form.

The ALDH-2 inhibitor compounds useful in the methods of the present disclosure, e.g., compound of Formula (I) such as compound (2), are effective over a wide dosage range and is generally administered as a pharmaceutical composition in a pharmaceutically effective amount. In some embodiments for oral administration, each dosage unit contains from about 10 mg to 1 g of an ALDH-2 inhibitor compound, such as compound of Formula (I), in some embodiments from 10 mg to 700 mg. In some embodiments, for parenteral administration, from 10 to 700 mg of an ALDH-2 inhibitor compound, such as compound of Formula (I), or in some embodiments, from about 50 mg to 300 mg.

Generally, in the methods of the disclosure, the amount of the ALDH-2 inhibitor compound, such as compound of Formula (I), to be administered in combination with the varenicline will be determined by a physician, in view of relevant circumstances of the subject being so treated, the chosen route of administration, and of course, the age, the weight, the severity of symptoms, the response of the individual subject to the treatment, and the like.

For preparing a solid pharmaceutical composition useful in the methods of the present disclosure, the active ingredient(s) is mixed with a pharmaceutical excipient to form a solid preformulation composition containing a homogeneous mixture of the active ingredient(s) and the excipients. When referring to these preformulation compositions as homogeneous, it is meant that the active ingredient(s) is dispersed evenly throughout the composition so that the composition may be readily subdivided into equally effective unit dosage forms such as tablets, pills and capsules. Tablets or pills may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Another exemplary mode for administering useful in the methods of the present disclosure is parenteral, particularly by injection. Pharmaceutical compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the active ingredients of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the known methods of preparation include vacuum-drying and freeze-drying techniques which yield a powder of the active ingredient plus any additional desired ingredient from a previously sterile-filtered solution thereof.

Pharmaceutical compositions that can be administered by inhalation or insufflation include solutions and suspensions in pharmaceutically acceptable, aqueous organic solvents, or mixtures thereof, and powders. The liquid or solid compositions may contain suitable pharmaceutically acceptable excipients as described herein and as known in the art. In some embodiments, the pharmaceutical composition(s) of the varenicline and the ALDH-2 inhibitor can be administered by the oral or nasal respiratory route for local or systemic effect. In some embodiments, the pharmaceutical compositions are prepared in pharmaceutically acceptable solvents which can be nebulized by use of inert gases. These nebulized solutions can be inhaled directly from the nebulizing device or the nebulizing device may be attached to a face mask tent, or intermittent positive pressure breathing machine. In some embodiments, the pharmaceutical composition(s) useful in the methods can be in solution, suspension, or powder compositions and can be administered, orally or nasally, from devices that deliver the formulation in an appropriate manner.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of Compound (1)—2,6-dichloro-N-[4-(2-oxo-1,2-dihydro-pyridin-4-yl)-benzyl]-benzamide

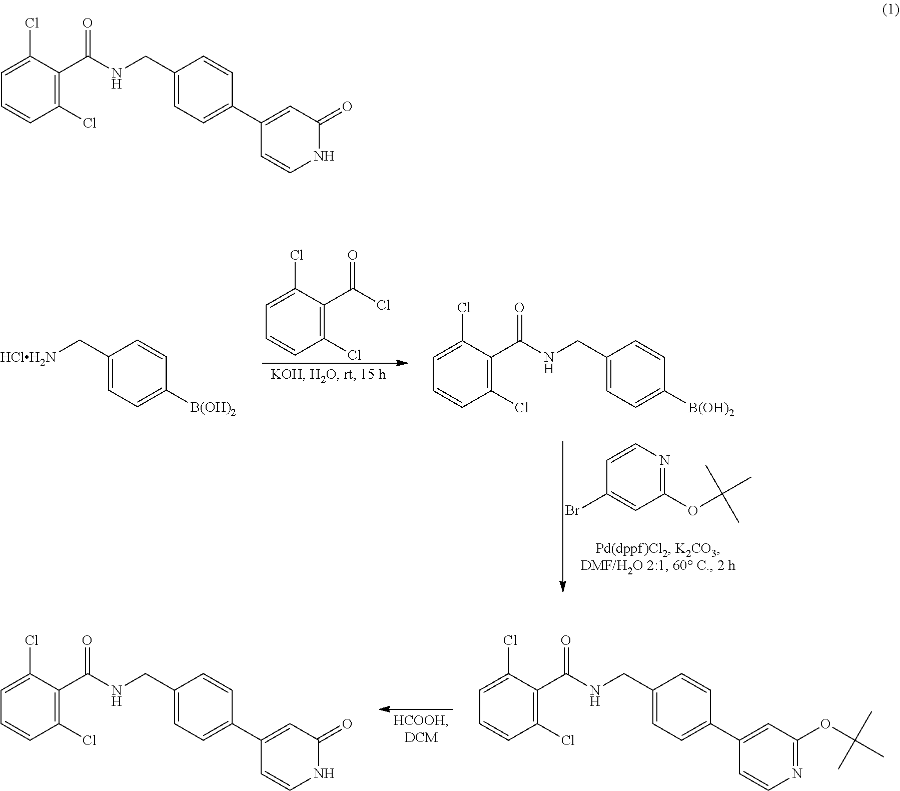

### Step 1—Preparation of 4-[(2,6-dichloro-benzoylamino)methyl]phenylboronic acid

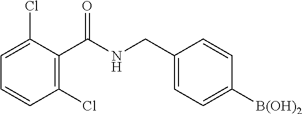 [SMILES: O=C(NCc1ccc(B(O)O)cc1)c1c(Cl)cccc1Cl]

4-(Aminomethyl)phenylboronic acid hydrochloride (5 g, 26.7 mmol) was dissolved in 25 mL water. 16 mL 50% aqueous KOH solution was added followed by 2,6-dichlorobenzoyl chloride (6.7 g, 32 mmol). The mixture was stirred rapidly at room temperature overnight. Acidification with 1N HCl gave a thick, white precipitate which was filtered, washed with water and dried giving 4-[(2,6-dichloro-benzoylamino) methyl]phenylboronic acid as a white powder in quantitative yield.

### Step 2—Preparation of N-[4-(2-tert-butoxy-pyridin-4-yl)-benzyl]-2,6-dichloro-benzamide

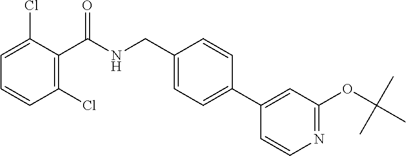 [SMILES: CC(C)(C)Oc1cc(-c2ccc(CNC(=O)c3c(Cl)cccc3Cl)cc2)ccn1]

4-[(2,6-Dichloro-benzoylamino)methyl]phenylboronic acid (5 g, 15.4 mmol), potassium carbonate (5 g), and [1,1′ bis(diphenylphosphino)ferrocene] dichloropalladium (II) (0.56 g, 0.77 mmol) were combined in a round bottom flask. 4-Bromo-2-(t-butoxy) pyridine (3.55 g, 15.4 mmol) was dissolved in 20 mL DMF and added to the flask under stirring. The flask was flushed with nitrogen and 10 mL water was added. The reaction mixture was stirred at 70° C. for two hours. After cooling the mixture was poured into 300 mL ethyl acetate and washed with water and brine. The organic phase was dried with magnesium sulfate and evaporated under vacuum. The crude N-[4-(2-tert-butoxy-pyridin-4-yl)-benzyl]-2,6-dichloro-benzamide was further purified by silica gel chromatography (eluent: hexane/ethyl acetate 1:1).

### Step 3—Preparation of 2,6-Dichloro-N-[4-(2-oxo-1,2-dihydro-pyridin-4-yl)-benzyl]-benzamide

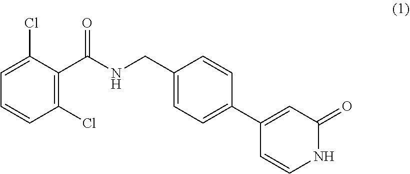 [SMILES: O=C(NCc1ccc(-c2cc[nH]c(=O)c2)cc1)c1c(Cl)cccc1Cl]

N-[4-(2-tert-Butoxy-pyridin-4-yl)-benzyl]-2,6-dichloro-benzamide was dissolved in 30 mL dichloromethane and 12 mL of 98% formic acid. The mixture was stirred at 40° C. for three hours after which the volatile components were evaporated under vacuum. The residue was triturated with ethyl acetate, filtered, washed with ethyl acetate and dried giving 2,6-dichloro-N-[4-(2-oxo-1,2-dihydro-pyridin-4-yl)-benzyl]-benzamide (4.34 g, 75.5% yield over two steps) as white powder. C₁₉H₁₄Cl₂N₂O₂: MS m/z: 373 (MH⁺) ¹H NMR (DMSO-d₆): δ 11.56 (s, 1H), δ 9.21 (t, J=5.6 Hz, 1H), δ 7.67 (d, J=8.0 Hz, 2H), δ 7.46 (m, 6H), δ 6.57 (d, J=1.2 Hz, 1H), δ 6.49 (dd, J=6.8 Hz, J′=1.6 Hz, 1H), δ 4.50 (d, J=6.0 Hz, 2H.

### Example 2: Preparation of Compound (2)—phosphoric acid mono-(4-{4-[(2,6-dichloro-benzoylamino)-methyl]-phenyl}-2-oxo-2H-pyridin-1-ylmethyl) ester

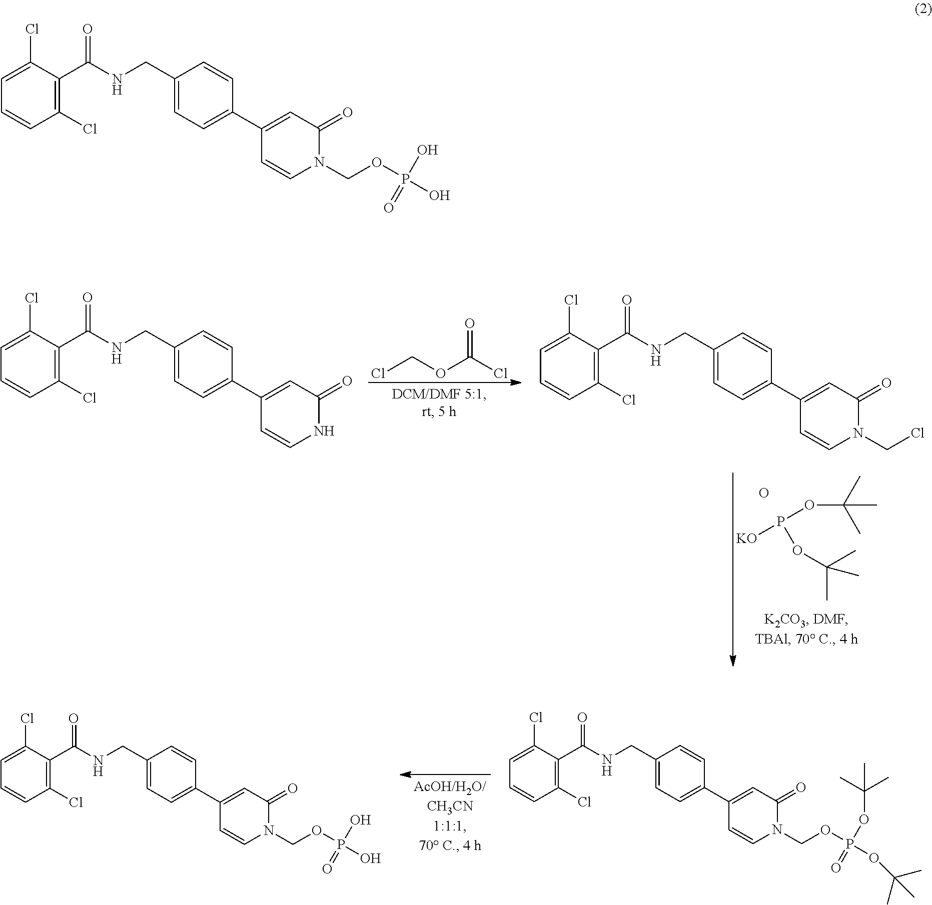

### Step 1—Preparation of 2,6-dichloro-N-[4-(1-chloromethyl-2-oxo-1,2-dihydro-pyridin-4-yl)-benzyl]-benzamide

2,6-Dichloro-N-[4-(2-oxo-1,2-dihydro-pyridin-4-yl)-benzyl]-benzamide (1.62 g, 4.34 mmol) (compound (1)), was suspended in 15 mL dichloromethane. Chloromethyl chloroformate (0.672 g, 5.21 mmol) was added followed by 3 mL DMF. The mixture was stirred at room temperature for five hours. After diluting with 200 mL ethyl acetate, the organic phase was washed with saturated, aqueous sodium bicarbonate solution and brine, dried with magnesium sulfate and evaporated under vacuum. The crude 2,6-dichloro-N-[4-(1-chloromethyl-2-oxo-1,2-dihydro-pyridin-4-yl)-benzyl]-benzamide was used in the following step without further purification.

### Step 2—Preparation of phosphoric acid di-tert-butyl ester 4-{4-[(2,6-dichloro-benzoylamino)-methyl]-phenyl}-2-oxo-2H-pyridin-1-ylmethyl ester

2,6-Dichloro-N-[4-(1-chloromethyl-2-oxo-1,2-dihydro-pyridin-4-yl)-benzyl]-benzamide from the previous step was dissolved in 50 mL DMF. Potassium carbonate (1 g) was added followed by potassium di(t-butyl)phosphate (2 g) and tetrabutylammonium iodide (50 mg). The mixture was stirred at 70° C. for four hours after which it was poured into 300 mL ethyl acetate. The organic phase was washed with water and brine, dried with magnesium sulfate and evaporated under vacuum. The crude product was further purified by silica gel chromatography (eluent: ethyl acetate), giving phosphoric acid di-tert-butyl ester 4-{4-[(2,6-dichloro-benzoylamino)-methyl]-phenyl}-2-oxo-2H-pyridin-1-ylmethyl ester as a colorless oil which slowly crystallized.

### Step 3—Preparation of phosphoric acid mono-(4-{4-[(2,6-dichloro-benzoylamino)-methyl]-phenyl}-2-oxo-2H-pyridin-1-ylmethyl) ester

Phosphoric acid di-tert-butyl ester 4-{4-[(2,6-dichloro-benzoylamino)-methyl]-phenyl}-2-oxo-2H-pyridin-1-ylmethyl ester from the previous step was dissolved in 20 mL acetonitrile, 20 mL acetic acid and 20 mL water, and heated at 70° C. for four hours. All volatile components were evaporated under vacuum and the residue was dissolved in 10 mL DMF. Slow addition of acetonitrile (˜60 mL) precipitated the product which was filtered, washed with more acetonitrile and dried, giving phosphoric acid mono-(4-{4-[(2,6-dichloro-benzoylamino)-methyl]-phenyl}-2-oxo-2H-pyridin-1-ylmethyl) ester (1.17 g, 56% over three steps) as a white powder. ¹H-NMR (DMSO) δ: 9.23 (t, J=6.2 Hz, 1H), 7.73 (d, J=8.4 Hz, 2H), 7.71 (d, J=8.4 Hz, 1H), 7.52-7.40 (m, 5H), 6.72 (d, J=1.6 Hz, 1H), 6.65 (dd, J=7.2 Hz, J=1.6 Hz, 1H), 5.61 (d, J=9.6 Hz, 2H), 4.52 (d, J=6.4 Hz, 2H). MS: 483/485 (MH⁺).

### Example 3: Formulation of Pharmaceutical Compositions

This example illustrates formulations of the pharmaceutical compositions comprising ALDH-2 inhibitor of formula (I) and varenicline and that can be used in the methods of the present disclosure for reducing nicotine addiction or aiding in the cessation or lessening of tobacco use.

Hard gelatin capsules: The ingredients listed below are mixed and filled into hard gelatin capsules:

| | Quantity |
|---|---|
| Ingredient | (mg/capsule) |
| Active Ingredient | 30.0 |
| Starch | 305.0 |
| Magnesium stearate | 5.0 |

240 mg Tablets: The ingredients listed below are blended and compressed to form 240 mg tablets:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

120 mg Tablets: The ingredients listed below are blended and compressed as described below to form 120 mg tablets:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |
| Total | 120 | mg |

The active ingredient, starch, and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

Suppositories: Suppositories each containing 25 mg of active ingredient, are made as follows:

| Ingredient | Quantity | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Suspensions: A suspension containing 50 mg of active ingredient per 5.0 mL dose, is made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Subcutaneous: a subcutaneous formulation is prepared as follows:

| Ingredient | Quantity |
|---|---|
| Active Ingredient | 5.0 mg |
| Corn Oil | 1.0 mL |

Injectable: an injectable formulation is prepared by combining the following ingredients:

| Ingredient | Quantity | |
|---|---|---|
| Active ingredient | 2.0 | mg/mL |
| Mannitol, USP | 50 | mg/mL |

| | |
|---|---|
| Gluconic acid, USP | q.s. (pH 5-6) |
| Water (distilled, sterile) | q.s. to 1.0 mL |
| Nitrogen Gas, NF | q.s. |

Topical: a topical preparation is prepared by combining the following ingredients as described below:

| Ingredients | Quantity (g) |
|---|---|
| Active ingredient | 0.01-1 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### Example 4: Determination of Combination Therapy Dosages to Reduce Nicotine Addiction in a Preclinical Animal Model

This example illustrates a preclinical animal model study of nicotine addiction for evaluating dose combinations of the ALDH-2 inhibitor of compound (2) and varenicline useful for enhancing tobacco cessation while reducing the quantity of varenicline and/or compound (2) administered to optimal levels for reducing side-effect risks.

Experimental Design and Protocol: Young adult male Sprague-Dawley rats are fitted with intravascular (IV) jugular infusion catheters and trained to self-administer nicotine during 13 consecutive days of self-administration training sessions of 45 minutes using an automated operant box equipped with two identical levers. Pressing the active lever results in an IV infusion of 50 μL nicotine coincident with light and sound cues. Pressing the inactive lever results in no infusion or following cues, but each is recorded. During the first five days of training, each active lever press during the time-in period results in the delivery of an infusion (FR-1). The response requirement then is increased to two active lever presses (i.e., FR-2) for 3 days, and finally to a value of five active lever presses (i.e., FR-5) for 5 days.

After acclimation, training, and acquisition of a stable nicotine self-administration response, rats are administered by oral gavage of one of the following: (i) vehicle alone; (ii) compound (2) alone (5 mg/kg or 10 mg/kg); (iii) varenicline alone (1.5 mg/kg or 3 mg/kg); (iv) combination of compound (2) (5 mg/kg or 10 mg/kg) and varenicline (1.5 mg/kg or 3 mg/kg) combined. The test compounds are administered 1 h before the nicotine plus cue self-administration (n=13-14/group).

Results: This study can show the dosage combination of the ALDH-2 inhibitor of compound (2) and varenicline that is most effective in reducing nicotine self-administration. The study also can show the extent to which a low dose of varenicline in combination with the ALDH-2 inhibitor of compound (2) provides a greater effect on reducing nicotine self-administration than each drug given alone, thereby indicating a beneficial synergistic effect of the combination.

### Example 5: Determining Effect of Combination Therapy on Progressive-Ratio Schedule of Intravenous Nicotine Self-Administration in a Preclinical Animal Model

This example illustrates a preclinical animal model study using a progressive-ratio schedule of intravenous nicotine self-administration to further assess the effect of the ALDH-2 inhibitor of compound (2) in combination with varenicline to reduce motivation for nicotine self-administration.

Experimental Design and Protocol: After self-administration training under FR-5 as described in Example 4, young adult male Sprague-Dawley rats are switched to a progressive ratio (PR) schedule where the number of active lever pressings required (response requirement) to receive an infusion of 50 μL nicotine is increased with each successive injection or food pellet delivery. The progression of active lever presses to meet the response requirement is 5, 10, 17, 24, 32, 42, 56, 73, 95, 124, 161, 208. The break point (BP) is defined as the highest ratio completed prior to the first 30 min period without a response on the active lever. Sessions under the PR schedule are set at a maximum time of 4 h. Animals are allowed 10-15 days of training depending upon time of stabilization of nicotine or food self-administration on the PR schedule before beginning administration of the test compounds. Test compounds or vehicle are administered by oral gavage 2 h prior to session start as follows: vehicle; compound (2) (5 and 10 mg/kg); varenicline (1.5 and 3 mg/kg); or the combination of compound (2) and varenicline.

Results: The results of the study can show the extent to which the ALDH-2 inhibitor of compound (2) administered in combination with varenicline is effective in reducing the number of nicotine infusions when each subsequent infusion requires progressively more work by the animal. The study also can show the extent to which a low dose of varenicline in combination with the ALDH-2 inhibitor of compound (2) provides a greater effect on reducing the nicotine break-point under a progressive ratio schedule of nicotine infusions, and thereby indicating a beneficial synergistic effect of the combination.

### Example 6: Determining Effect of Combination Therapy on Cue-Induced Reinstatement of Nicotine-Seeking Behavior in a Preclinical Animal Model

This example illustrates a preclinical animal model study using intravenous nicotine self-administration together with an extinction period to assess the effect of the ALDH-2 inhibitor of compound (2) in combination with varenicline to reduce cue-induced reinstatement of nicotine seeking behavior.

Experimental Design and Protocol: After self-administration training under FR-5 as described in Example 4, subsequent self-administration sessions of the young adult male Sprague-Dawley rats are conducted without any nicotine present in order to extinguish drug-seeking behavior. The criterion for extinction is less than 20 active lever presses per 1 h session over two consecutive days. After stable extinction is achieved, cue-induced reinstatement testing of the rats is conducted under conditions identical to those of self-administration sessions described in Example 4 except that: (1) a single presentation of the cues (i.e., light above the active lever on and house-light off for 1 min) is delivered immediately at the start of the session; (2) responses on the active lever (on an FR-5 schedule) result in continued presentation of the cues without nicotine availability (i.e., no injections); (3) each reinstatement testing session lasts 1 h; and (4) test compounds or vehicle are administered by oral gavage 2 h prior to testing session start as follows: vehicle; compound (2) (5 and 10 mg/kg); varenicline (1.5 and 3 mg/kg); or the combination of compound (2) and varenicline.

Results: The results of the study can show the extent to which the ALDH-2 inhibitor of compound (2) administered in combination with varenicline is effective in reducing the number of lever presses upon reinstatement of the conditioned response in the absence of an exposure to nicotine. The study can show the effect of low dose compound (2) in combination in low dose varenicline is greater than the additive effect of each drug alone, indicating a beneficial synergistic effect in reducing cue-induced behavior to seek nicotine.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the inventions.

What is claimed is:

1. A method for reducing nicotine addiction or aiding in the cessation or lessening of tobacco use in a mammal, comprising administering to said mammal a therapeutically effective amount of varenicline in combination with a therapeutically effective amount of an ALDH-2 inhibitor.

2. The method of claim 1, wherein the therapeutically effective amount of varenicline is
(a) less than 2.0 mg, less than 1.0 mg, less than 0.5 mg, less than 0.25 mg, less than 0.1 mg, or less than 0.05 mg;
(b) between about 0.05 mg and 2.0 mg, between about 0.05 mg and 1.0 mg, between about 0.05 mg and 0.5 mg, between about 0.05 mg and 0.4 mg, between about 0.05 mg and 0.25 mg, or between about 0.05 mg and 0.15 mg; or
(c) less than 2.0 mg/day, less than 1.0 mg/day, less than 0.5 mg/day, less than 0.25 mg/day, or less than 0.1 mg/day.

3. The method of claim 1, wherein the therapeutically effective amount of ALDH-2 inhibitor is
(a) less than 5 mg, less than 2.5 mg, less than 1.0 mg, or less than 0.5 mg;
(b) between 0.5 mg and 5 mg, between about 0.5 mg and 4.0 mg, between about 0.5 mg and 2.5 mg, or between about 0.5 mg and 1.5 mg; or
(c) less than 5 mg/day, less than 2.5 mg/day, or less than 1.0 mg/day.

4. The method of claim 2, wherein the therapeutically effective amount of ALDH-2 inhibitor is
(a) less than 5 mg, less than 2.5 mg, less than 1.0 mg, or less than 0.5 mg;
(b) between 0.5 mg and 5 mg, between about 0.5 mg and 4.0 mg, between about 0.5 mg and 2.5 mg, or between about 0.5 mg and 1.5 mg; or
(c) less than 5 mg/day, less than 2.5 mg/day, or less than 1.0 mg/day.
